(19) United States
(12) Patent Application Publication
(10) Pub. No.: US 2003/0125352 A1
(43) Pub. Date: Jul. 3, 2003

(54) Therapeutic tropane compounds

(76) Inventors: Bertha K. Madras, Newton, MA (US); Peter C. Meltzer, Lexington, MA (US); Paul Blundell, Winchester, MA (US)

Correspondence Address: Dike, Bronstein, Roberts & Cushman, Intellectual Property Practice Group, EDWARDS & ANGELL, LLP, P.O. Box 9169, Boston, MA, 02209, US

(21) Appl. No.: 10/222,530

(22) Filed: Aug. 16, 2002

Related U.S. Application Data
(60) Provisional application No. 60/313,205, filed on Aug. 17, 2001.

(51) Int. Cl. A61K 31/46

(52) U.S. Cl. 514/304

(57) ABSTRACT

This invention relates to therapeutic uses of boat tropane analogs, e.g, treatment of neurodegenerative disorders. More specifically the invention relates to a method of treating a neurological disorder in patient comprising administering to the patient an effective amount of a boat tropane compound.

### RELATED APPLICATION

This application claims the benefit of U.S. provisional patent application Serial No. 60/313,205, filed Aug. 17, 2001, entitled “Therapeutic Tropane Compounds,” the entire teachings of which are incorporated herein by reference.

### STATEMENT OF GOVERNMENT SUPPORT

This invention was made with government support under Grant Numbers DA 11542, DA 7-8081, DA 1-8825, DA06303, DA 00304 and RR 00168 awarded by the NIH/NIDA. The government has certain rights in the invention.

### FIELD OF THE INVENTION

This invention relates to therapeutic uses of boat tropane analogs, e.g., treatment of neurodegenerative disorders.

### BACKGROUND OF THE INVENTION

The dopamine transporter (DAT) plays a critical role in physiological, pharmacological and pathological processes in brain. The transport system is a primary mechanism for terminating the effects of synaptic dopamine, thereby contributing to the maintenance of homeostasis in dopamine systems. It also appears to be a principal target of cocaine in the brain. (Kennedy and Hanbauer, J. Neurochem. 1983, 41, 172-178; Shoemaker et al., Naunyn-Schmeideberg's Arch. Pharmacol. 1985, 329, 227-235; Reith et al., Biochem Pharmacol. 1986, 35, 1123-1129; Ritz et al., Science 1987, 237, 1219-1223; Madras et al., J. Pharmacol. Exp. Ther. 1989a, 251, 131-141; Bergman et al., J. Pharmacol. Exp. Ther. 1989, 251, 150-155; Madras and Kaufman, Synapse 1994, 18, 261-275). Furthermore, the dopamine transporter may be a conduit for entry of neurotoxins into dopamine containing cells.

The striatum has the highest levels of dopamine terminals in the brain. A high density of DAT is localized on dopamine neurons in the striatum and appears to be a marker for a number of physiological and pathological states. For example, in Parkinson's disease, dopamine is severely reduced and the depletion of DAT in the striatum has been an indicator for Parkinson's disease (Schoemaker et al., Naunyn-Schmeideberg's Arch. Pharmacol. 1985, 329, 227-235; Kaufman and Madras, Synapse 1991, 9, 43-49). Consequently, early or presymptomatic diagnosis of Parkinson's disease can be achieved by the quantitative measurement of DAT depletion in the striatum. (Kaufman and Madras, Synapse 1991, 9, 43-49).

Other neuropsychiatric disorders, including Tourette's Syndrome and Lesch Nyhan Syndrome and possibly Rett's syndrome, are also marked by changes in DAT density. The DAT also is the target of the most widely used drug for Attention Deficit Disorder, methylphenidate. Other diseases, e.g., depression, can be affected. See Diagnostic and Statistical Manual of Mental Disorders-IV (DSM-IV), the contents of which are incorporated by reference. Furthermore, an age-related decline in dopamine neurons can be reflected by a decline in the dopamine transporter (Kaufman and Madras, Brain Res. 1993, 611, 322-328; van Dyck et al., J. Nucl. Med. 1995, 36, 1175-1181) and may provide a view on dopamine deficits that lie outside the realm of neuropsychiatric diseases.

The density of the DAT in the brains of substance abusers has also been shown to deviate from that in normal brain. For example, the density is elevated in post-mortem tissues of cocaine abusers (Little et al., Brain Res. 1993, 628, 17-25). On the other hand, the density of the DAT in chronic nonviolent alcohol abusers is decreased markedly. (Tiihonen et al., Nature Medicine 1995, 1, 654-657). Cocaine dependence is a problem of national significance. To date no cocaine pharmacotherapy has been reported. Cocaine is a potent stimulant of the mammalian central nervous system. Its reinforcing properties and stimulant effects are associated with its propensity to bind to monoamine transporters, particularly the dopamine transporter (DAT). (Kennedy, L. T. and I. Hanbauer (1983), J. Neurochem. 34: 1137-1144; Kuhar, M. J., M. C. Ritz and J. W. Boja (1991), Trends Neurosci. 14: 299-302; Madras, B. K., M. A. Fahey, J. Bergman, D. R. Canfield and R. D. Spealman (1989), J. Pharmacol. Exp. Ther. 251: 131-141; Madras, B. K., J. B. Kamien, M. Fahey, D. Canfield, et al. (1990), Pharmacol Biochem. Behav. 35: 949-953; Reith, M. E. A., B. E. Meisler, H. Sershen and A. Lajtha (1986), Biochem. Pharmacol. 35: 1123-1129; Ritz, M. C., R. J. Lamb, S. R. Goldberg and M. J. Kuhar (1987), Science 237: 1219-1223; Schoemaker, H., C. Pimoule, S. Arbilla, B. Scatton, F. Javoy-Agid and S. Z. Langer (1985), Naunyn-Schmiedeberg's Arch. Pharmacol. 329: 227-235.) It also binds with substantial potency to serotonin transporters (SERT) and norepinephrine transporters.

Structure activity relationship (SAR) studies have largely focused on a series of cocaine analogs. Among the more potent of these congeners at ³H-cocaine binding sites in striatum (Madras, B. K., M. A. Fahey, J. Bergman, D. R. Canfield and R. D. Spealman (1989), J Pharmacol. Exp. Ther. 251: 131-141; Reith, M. E. A., B. E. Meisler, H. Sershen and A. Lajtha (1986), Biochem. Pharmacol. 35: 1123-1129) is (1R)-3β-(4-fluorophenyl)tropane-2β-carboxylic acid methyl ester, (WIN35,428 or CFT) (Kaufman, M. J. and B. K. Madras (1992), Synapse 12: 99-111; Madras, B. K., M. A. Fahey, J. Bergman, D. R. Canfield and R. D. Spealman (1989), J. Pharmacol. Exp. Ther. 251: 131-141) reported in 1973 (Clarke, R. L., S. J. Daum, A. J. Gambino, M. D. Aceto, et al. (1973), J. Med. Chem. 16: 1260-1267). This compound was subsequently radiolabeled to provide a selective probe for the DAT in primate brain. (Canfield, D. R., R. D. Spealman, M. J. Kaufman and B. K. Madras (1990), Synapse 6: 189-195; Kaufman, M. J. and B. K. Madras (1991), Synapse 9: 43-49; Kaufman, M. J., R. D. Spealman and B. K. Madras (1991), Synapse 9: 177-187.)

Accordingly, a pharmaceutical that binds to the DAT can assist in the treatment of these various disease states.

Among the most potent tropane inhibitors of monoamine binding sites in striatum are 3β-{4-(1-methylethenyl)-phenyl}-2β-propanoyl-8-azabicyclo (3.2.1)octane and 3β-(2-naphthyl)-2β-propanoyl-8-azabicyclo(3.2.1)octane, (Bennett, B. A., C. H. Wichems, C. K. Hollingsworth, H. M. L. Davies, C. Thornley, T. Sexton and S. R. Childers (1995), J. Pharm. Exp. Ther. 272: 1176-1186; Davies, H. M. L., L. A. Kuhn, C. Thornley, J. J. Matasi, T. Sexton and S. R. Childers (1996), J Med. Chem. 39: 2554-2558) (1R)-RI55 (βCIT), (Boja 1991; Boja, J. W., A. Patel, F. I. Carroll, M. A. Rahman, et al. (1991), Eur. J. Pharmacol. 194: 133-134; Neumeyer, J. L., S. Wang, R. A. Milius, R. M. Baldwin, et al. (1991), J. Med. Chem. 34: 3144-3146) (1R)-RTI121, (Carroll, F. I., A. H. Lewin, J. W. Boja and M. J. Kuhar (1992), J. Med. Chem. 35: 969-981.) and (1R)-3β-(3,4-di-chlorophenyl)-tropane-2β-carboxylic acid methyl ester (O-401), (Carroll, F. I., M. A. Kuzemko and Y. Gao (1992), Med. Chem Res. 1: 382-387; Meltzer, P. C., A. Y. Liang, A.-L. Brownell, D. R. Elmaleh and B. K. Madras (1993), J. Med. Chem. 36: 855-862).

SAR studies of the binding of these agents and their effects on monoamine transporter function have been reported. (Blough, B. E., P. Abraham, A. H. Lewin, M. J. Kuhar, J. W. Boja and F. I. Carroll (1996), J. Med. Chem. 39: 4027-4035; Carroll, F. I., P. Kotian, A. Dehghani, J. L. Gray, et al. (1995), J. Med. Chem. 38: 379-388; Carroll, F. I., A. H. Lewin, J. W. Boja and M. J. Kuhar (1992),y J. Med. Chem. 35: 969-981; Carroll, F. I., S. W. Mascarella, M. A. Kuzemko, Y. Gao, et al. (1994), J. Med. Chem. 37: 2865-2873; Chen, Z., S. Izenwasser, J. L. Katz, N. Zhu, C. L. Klein and M. L. Trudell (1996), J. Med. Chem. 39: 4744-4749; Davies, H. M. L., L. A. Kuhn, C. Thornley, J. J. Matasi, T. Sexton and S. R. Childers (1996), J. Med. Chem. 39: 2554-2558; Davies, H. M. L., Z.-Q. Peng and J. H. Houser (1994), Tetrahedron Lett. 48: 8939-8942; Davies, H. M. L., E. Saikali, T. Sexton and S. R. Childers (1993), Eur. J. Pharmacol. Mol. Pharm. 244: 93-97; Holmquist, C. R., K. I. Keverline-Frantz, P. Abraham, J. W. Boja, M. J. Kuhar and F. I. Carroll (1996), J. Med. Chem 39: 4139-4141; Kozikowski, A. P., G. L. Araldi and R. G. Ball (1997), J. Org. Chem. 62: 503-509; Kozikowski, A. P., M. Roberti, L. Xiang, J. S. Bergmann, P. M. Callahan, K. A. Cunningham and K. M. Johnson (1992), J. Med. Chem. 35: 4764-4766; Kozikowski, A. P., D. Simoni, S. Manfredini, M. Roberti and J. Stoelwinder (1996), Tetrahedron Lett. 37: 5333-5336; Meltzer, P. C., A. Y. Liang, A.-L. Brownell, D. R. Elmaleh and B. K. Madras (1993), J. Med. Chem. 36: 855-862; Meltzer, P. C., A. Y. Liang and B. K. Madras (1994), J. Med. Chem. 37: 2001-2010; Meltzer, P. C., A. Y. Liang and B. K. Madras (1996), J. Med. Chem. 39: 371-379; Newman, A. H., A. C. Allen, S. Izenwasser and J. L. Katz (1994), J. Med Chem. 37: 2258-2261; Newman, A. H., R. H. Kline, A. C. Allen, S. Izenwasser, C. George and J. L. Katz (1995), J. Med. Chem. 38: 3933-3940; Shreekrishna, V. K., S. Izenwasser, J. L. Katz, C. L. Klein, N. Zhu and M. L. Trudell (1994), J. Med. Chem. 37: 3875-3877; Simoni, D., J. Stoelwinder, A. P. Kozikowski, K. M. Johnson, J. S. Bergmann and R. G. Ball (1993), J. Med. Chem. 36: 3975-3977.)

Binding of cocaine and its tropane analogs to monoamine transporters is stereoselective. As example (1R)-(−)-cocaine binds at the dopamine transporter about 200-fold more potently than the unnatural isomer, (1S)-(+)-cocaine. (Kaufman, M. J. and B. K. Madras (1992), Synapse 12: 99-111; Madras, B. K., M. A. Fahey, J. Bergman, D. R. Canfield and R. D. Spealman (1989), J. Pharmacol. Exp. Ther. 251: 131-141; Madras, B. K., R. D. Spealman, M. A. Fahey, J. L. Neumeyer, J. K. Saha and R. A. Milius (1989), Mol. Pharmacol. 36: 518-524; Reith, M. E. A., B. E. Meisler, H. Sershen and A. Lajtha (1986), Biochem. Pharmacol. 35: 1123-1129; Ritz, M. C., R. J. Lamb, S. R. Goldberg and M. J. Kuhar (1987), Science 237: 1219-1223.)

Also, only the R-enantiomers of cocaine have been found active in a variety of biological and neurochemical measures. (Clarke, R. L., S. J. Daum, A. J. Gambino, M. D. Aceto, et al. (1973), J. Med. Chem. 16: 1260-1267; Kaufman, M. J. and B. K. Madras (1992), Synapse 12: 99-111; Madras, B. K., M. A. Fahey, J. Bergman, D. R. Canfield and R. D. Spealman (1989), J. Pharmacol. Exp. Ther. 251: 131-141; Madras, B. K., R. D. Spealman, M. A. Fahey, J. L. Neumeyer, J. K. Saha and R. A. Milius (1989), Mol. Pharmacol. 36: 518-524; Reith, M. E. A., B. E. Meisler, H. Sershen and A. Lajtha (1986), Biochem. Pharmacol. 35: 1123-1129; Ritz, M. C., R. J. Lamb, S. R. Goldberg and M. J. Kuhar (1987), Science 237: 1219-1223; Sershen, H., M. E. A. Reith and A. Lajtha (1980), Neuropharmacology 19: 1145-1148; Sershen, H., M. E. A. Reith and A. Lajtha (1982), Neuropharmacology 21: 469-474; Spealman, R. D., R. T. Kelleher and S. R. Goldberg (1983), J. Pharmacol. Exp. Ther. 225: 509-513.) Parallel stereoselective behavioral effects have also been observed. (Bergman, J., B. K. Madras, S. E. Johnson and R. D. Spealman (1989), J. Pharmacol. Exp. Ther. 251: 150-155; Heikkila, R. E., L. Manzino and F. S. Cabbat (1981), Subst. Alcohol Actions/Misuse 2: 115-121; Reith, M. E. A., B. E. Meisler, H. Sershen and A. Lajtha (1986), Biochem. Pharmacol. 35: 1123-1129; Spealman, R. D., R. T. Kelleher and S. R. Goldberg (1983), J. Pharmacol. Exp. Ther. 225: 509-513; Wang, S., Y. Gai, M. Laruelle, R. M. Baldwin, B. E. Scanlet, R. B. Innis and J. L. Neumeyer (1993), J. Med. Chem. 36: 1914-1917.) For example, in primates and rodents the stimulating and reinforcing properties of the (−)-enantiomer of cocaine or its 3-aryltropane analogs were considerably greater than for the (+)-enantiomers.

Although SAR studies of cocaine and its 3-aryltropane analogs have offered insight into their mode of binding to monoamine transporters, a comprehensive picture of the binding interaction at the molecular level has not emerged. SAR studies on the classical tropane analogs (Carroll, F. I., Y. Gao, M. A. Rahman, P. Abraham, et al. (1991), J. Med. Chem. 34: 2719-2725; Carroll, F. I., S. W. Mascarella, M. A. Kuzemko, Y. Gao, et al. (1994), J. Med. Chem. 37: 2865-2873; Madras, B. K., M. A. Fahey, J. Bergman, D. R. Canfield and R. D. Spealman (1989), J. Pharmacol. Exp. Ther. 251: 131-141; Madras, B. K., R. D. Spealman, M. A. Fahey, J. L. Neumeyer, J. K. Saha and R. A. Milius (1989), Mol. Pharmacol. 36: 518-524; Meltzer, P. C., A. Y. Liang, A.-L. Brownell, D. R. Elmaleh and B. K. Madras (1993), J. Med. Chem. 36: 855-862; Reith, M. E. A., B. E. Meisler, H. Sershen and A. Lajtha (1986), Biochem. Pharmacol. 35: 1123-1129) appeared to provide a consistent model for this interaction with the DAT, however, subsequent studies revealed inconsistencies. (Carroll, F. I., P. Kotian, A. Dehghani, J. L. Gray, et al. (1995), J. Med. Chem. 38: 379-388; Chen, Z., S. Izenwasser, J. L. Katz, N. Zhu, C. L. Klein and M. L. Trudell (1996), J. Med. Chem. 39: 4744-4749; Davies, H. M. L., L. A. Kuhn, C. Thornley, J. J. Matasi, T. Sexton and S. R. Childers (1996), J. Med. Chem. 39: 2554-2558; Kozikowski, A. P., G. L. Araldi and R. G. Ball (1997), J. Org. Chem. 62: 503-509; Meltzer, P. C., A. Y. Liang and B. K. Madras (1994), J Med. Chem. 37: 2001-2010; Meltzer, P. C., A. Y. Liang and B. K. Madras (1996), J. Med. Chem. 39: 371-379.)

Carroll had proposed (Boja, J. W., R. M. McNeill, A. Lewin, P. Abraham, F. I. Carroll and M. J. Kuhar (1992), Mol. Neurosci. 3: 984-986; Carroll, F. I., P. Abraham, A. Lewin, K. A. Parham, J. W. Boja and M. J. Kuhar (1992), J. Med. Chem. 35: 2497-2500; Carroll, F. I., Y. Gao, M. A. Rahman, P. Abraham, et al. (1991), J. Med. Chem. 34: 2719-2725; Carroll, F. I., M. A. Kuzemko and Y. Gao (1992), Med. Chem Res. 1: 382-387) four molecular requirements for binding of cocaine and its tropane analogs at the DAT: a 2β-carboxy ester, a basic nitrogen capable of protonation at physiological pH, the R-configuration of the tropane and a 3β-aromatic ring at C₃. However, Davies (Davies, H. M. L., E. Saikali, T. Sexton and S. R. Childers (1993), Eur. J. Pharmacol. Mol. Pharm. 244: 93-97) later reported that introduction of 2β-ketones did not reduce potency. Kozikowski questioned the role of hydrogen bonding at the C₂ site because introduction of unsaturated and saturated alkyl groups (Kozikowski, A. P., M. Roberti, K. M. Johnson, J. S. Bergmann and R. G. Ball (1993), Bioorg. Med. Chem. Lett. 3: 1327-1332; Kozikowski, A. P., M. Roberti, L. Xiang, J. S. Bergmann, P. M. Callahan, K. A. Cunningham and K. M. Johnson (1992), J. Med. Chem. 35: 4764-4766) did not diminish binding. Further, the ionic bond between a protonated amine (at physiologically pH) and the presumed (Kitayama, S., S. Shimada, H. Xu, L. Markham, D. H. Donovan and G. R. Uhl (1993), Proc. Natl. Acad. Sci. U.S.A. 89: 7782-7785) aspartate residue on the DAT was questioned because reduction of nitrogen nucleophilicity (Kozikowski, A. P., M. K. E. Saiah, J. S. Bergmann and K. M. Johnson (1994), J. Med. Chem. 37(37): 3440-3442) by introduction of N-sulfones did not reduce binding potency.

It also has been reported (Madras, B. K., J. B. Kamien, M. Fahey, D. Canfield, et al. (1990), Pharmacol Biochem. Behav. 35: 949-953) that introduction of an alkyl or allyl group did not eliminate binding potency. An N-iodoallyl group on the tropane has provided potent and selective ligands for the DAT, and altropane is currently being developed as a SPECT imaging agent (Elmaleh, D. R., B. K. Madras, T. M. Shoup, C. Byon, et al. (1995), J. Nucl. Chem., 37 1197-1202 (1966); Fischman, A. J., A. A. Bonab, J. W. Babich, N. M. Alpert, et al. (1996), Neuroscience-Net 1, 00010, (1997). A ^{99m}technetium labeled compound, technepine, which binds potently and selectively to the DAT and provides excellent in vivo SPECT images has been reported. (Madras, B. K., A. G. Jones, A. Mahmood, R. E. Zimmerman, et al. (1996), Synapse 22: 239-246.) (Meltzer, P. C., Blundell, P., Jones, A. G., Mahmood, A., Garada, B. et al., J. Med. Chem., 40, 1835-1844, (1997). 2-Carbomethoxy-3-(bis(4-fluorophenyl)methoxy)tropanes have been reported (Meltzer, P. C., A. Y. Liang and B. K. Madras (1994), J. Med. Chem. 37: 2001-2010). The S-enantiomer, S)-(+)-2β-carbomethoxy-3α-(bis(4-fluorophenyl)methoxy)tropane (Difluoropine) was considerably more potent (IC₅₀: 10.9 nM) and selective (DAT v. SERT: 324) than any of the other seven isomers, including the R-enantiomers.

Drug therapies for cocaine abuse are needed. Also, there is a need for therapeutic and protective agents for neurodegenerative diseases such as Parkinson's disease and Alzheimer's disease as well as therapeutic agents for dopamine related dysfunction such as Attention Deficit Disorder (ADD and ADHD). Compounds that inhibit monoamine reuptake in the mammalian system are sought to provide such therapies.

Inhibition of 5-hydroxytryptamine reuptake has an effect on diseases mediated by 5HT receptors. Compounds that provide such inhibition can be useful, for example, as therapeutic anti-depressants.

Cocaine recognition sites are localized on monoamine transporters such as, for example, the dopamine transporter (DAT) and serotonin transporter (SERT). These transporters are localized, in turn, on monoamine nerve terminals. Compounds that bind to these sites can be useful as (i) probes for neuro-degenerative diseases (e.g., Parkinson's disease), (ii) therapeutic drugs for neurodegenerative diseases (e.g., Parkinson's and Alzheimer's disease), (iii) therapeutic drugs for dopamine dysfunction (e.g., Attention Deficit Disorder (ADD) or Attention Deficit Hyperactivity Disorder (ADHD)), (iv) treatment of psychiatric dysfunction (e.g., depression) and (v) treatment of clinical dysfunction (e.g., migraine).

Thus it would be useful to have compounds that can be used for therapeutic treatment of neurodegenerative diseases, e.g., Parkinson's and Alzheimer's disease, dopamine dysfunction, e.g., Attention Deficit Disorder (ADD) or Attention Deficit Hyperactivity Disorder (ADHD), Tourette's Syndrome, Lesch Nyhan Syndrome and possibly Rett's syndrome, psychiatric dysfunction, e.g., depression, and treatment of clinical dysfunction, e.g., migraine.

### SUMMARY OF THE INVENTION

The present invention relates to the discovery that tropane compounds having the “boat” configuration show surprisingly effective results in treating certain neurological diseases, e.g., neurodegenerative diseases such as Parkinson's Disease.

Thus, the present invention relates to therapeutic uses of boat tropane analogs. More specifically, the invention relates to methods of treating patients having neurodegenerative diseases, dopamine dysfunction and other DAT related diseases, comprising administering to the patient boat tropane compounds. Such diseases include, but are not limited to, e.g., Parkinson's and Alzheimer's disease, Attention Deficit Disorder (ADD) or Attention Deficit Hyperactivity Disorder (ADHD), Tourette's Syndrome, Lesch Nyhan Syndrome, Rett's syndrome, depression, narcolepsy and migraine. The methods also include therapies for smoking cessation.

More specifically, the invention relates to the use of topane compounds having the boat configuration, as described further below, for the treatment of these diseases.

The present invention provides pharmaceutical therapeutic compositions comprising the compounds formulated in a pharmaceutically acceptable carrier for use in the present methods.

Further, the invention provides a method for inhibiting 5-hydroxytryptamine reuptake of a monoamine transporter by contacting the monoamine transporter with a 5-hydroxy-tryptamine reuptake inhibiting (5-HT inhibiting) amount of a boat tropane compound. Inhibition of 5-hydroxytryptamine reuptake of a monoamine transporter in a mammal is provided in accord with the present invention by administering to the mammal a 5-HT inhibiting amount of a boat tropane compound in a pharmaceutically acceptable carrier. Preferred monoamine transporters for the practice of the present invention include the dopamine transporter, the serotonin transporter and the norepinephrine transporter.

The invention also provides a method for inhibiting dopamine reuptake of a dopamine transporter by contacting the dopamine transporter with a dopamine reuptake inhibiting amount of a boat tropane compound. Inhibition of dopamine reuptake of a dopamine transporter in a mammal is provided in accord with the present invention by administering to the mammal a dopamine inhibiting amount of a boat tropane compound in a pharmaceutically acceptable carrier.

The invention also relates to a method for treating a mammal having a disorder selected from neurodegenerative disease, psychiatric dysfunction, dopamine dysfunction, cocaine abuse and clinical dysfunction comprising administering to the mammal an effective amount of a compound of the present invention. In preferred methods, the compound has a 3α-group. In certain methods, the neurodegenerative disease is selected from Parkinson's disease and Alzheimer's disease. An example of a psychiatric disorder which can be treated by the present methods is depression.

The invention also relates to methods for treating dopamine related dysfunction in a mammal comprising administering to the mammal a dopamine reuptake inhibiting amount of a compound as described herein. In preferred methods, the compound is a boat tropane. An example of a dopamine related dysfunction is Attention deficit disorder.

Certain preferred compounds used in the present invention have a high selectivity for the DAT versus the SERT. Preferred compounds have an IC₅₀ SERT/DAT ratio of greater than about 10, preferably greater than about 30 and more preferably 50 or more. In addition, preferably the compounds have an IC₅₀ at the DAT of less than about 500 nM, preferably less than 60 nM, more preferably less than about 20, and most preferably less than about 10.

### BRIEF DESCRIPTION OF THE FIGURES

FIG. 1 is an illustration of a general scheme for preparation of 2-carbomethoxy tropanes (Scheme 1) comprising an aryl octene in accord with the present invention and subsequent preparation of 3α and 3β diasteriomers thereof.

FIG. 2 is an illustration of a general scheme for preparation of 2-ethylketo analogs compounds in accord with a preferred embodiment of the present invention.

FIG. 3 illustrates the absolute Configurations of (1R)-8a, (1R)-18a, (1S)-18a.

FIG. 4 illustrates a reaction scheme (Scheme 1) for the preparation of 2,3-Unsaturated Tropanes.

FIG. 5 illustrates a reaction scheme (Scheme 2) for the preparation of Bridge Oxygenated Tropanes.

FIG. 6 illustrates a reaction scheme (Scheme 3) for the preparation of Bridge Oxygenated 2-Keto Tropanes.

FIG. 7 illustrates a reaction scheme (Scheme 4) for the resolution of 8a, 15a and 18a.

FIG. 8 illustrates a reaction scheme (Scheme 5) for the inversion at C6 and C7.

### DETAILED DESCRIPTION OF THE INVENTION

In accord with the present invention, methods are provided for administering to a patient suffering from certain neurological diseases, an effective amount of a boat tropane analog. Compounds useful as therapeutic agents in the methods of the present invention include boat tropane compounds described in pending application U.S. Ser. No. 09/568,106, U.S. Pat. No. 6,171,576, which issued on Jan. 9, 2001, provisional application no. 60/313,205 and U.S. application Ser. No. 10/033,621. These applications and patents are incorporated in their entirety.

Preferred compounds for use in the methods of the present invention comprise tropane analogs that bind to monoamine transporters. Examples of useful compounds are represented by the following general structural formula:

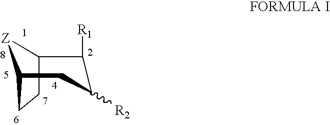

wherein

R₁ is α or β and is selected from COOR^{a}, COR^{a}, and CON(CH₃)OR^{a};

R₂ is α and is selected from C₆H₄X, C₆H₃XY, C₁₀H₇X, and C₁₀H₆XY;

R^{a} is selected from C₁-C₅ alkyl, e.g. methyl, ethyl, propyl, isopropyl, etc.;

X and Y are independently selected from R^{a}, H, Br, Cl, I, F, OH, and OCH₃;

Z=NR₃, NSO₂R_{3,} with R₃=H, (CH₂)ₙC₆H₄Y, C₆H₄Y, CHCH₂, lower alkyl lower alkenyl or lower alkynyl.

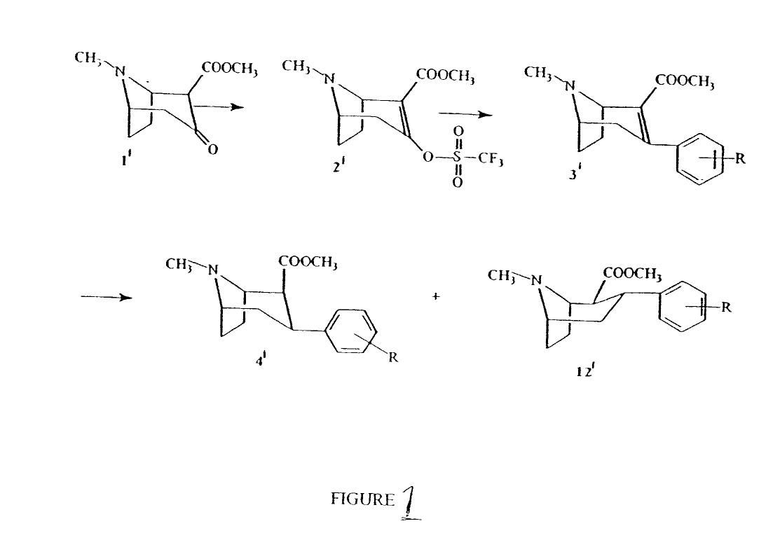

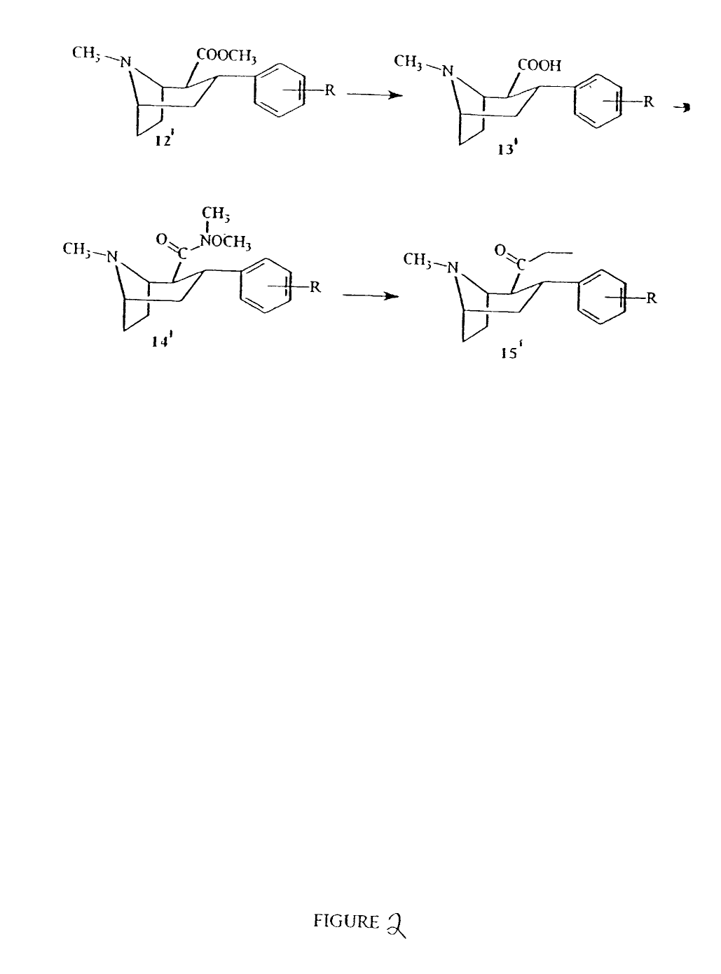

R₁ can be in the α or β configuration. R₂ is in the α configuration. Further, R₁ preferably can be substituted at the C₂ or C₄ when the tropane has a 1R or 1S configuration, respectively. Particularly preferred compounds comprise compound 15 shown in FIGS. 1 and 2, especially 2β-(1-Propanoyl)-3α-(4-fluorophenyl)-tropane, 2β-(1-Propanoyl)-3α-(3,4-dichlorophenyl)tropane.

Any tropane compound of the above general formula is useful in the present invention so long as it binds to DAT. Examples of particularly useful tropanes are: 2-carbomethoxy-3-(4-fluorophenyl)-N-methyltropane (“WIN 35,428”) (Clarke, R. L., et al., J. Med. Chem. 1973, 16, 1260-1267) which binds potently (IC₅₀=11.0 nM) and with specificity to the DAT (Meltzer, P. C., et al., J. Med. Chem. 1993, 36, 855-862); 2-carbomethoxy-3-(3,4-dichlorophenyl)-N -methyltropane (“O-401”; IC₅₀=1.09nM) (Meltzer, P. C., et al., J. Med. Chem. 1993, 36, 855-862). Tropane analogs that have a 3α-group are of the boat configuration. Other tropanes having a 3β-oriented group are of the chair configuration. Preferred compounds for use in the method of the present invention have the boat configuration.

Other compounds useful for treating neurological disorders include the boat tropanes disclosed in U.S. Pat. No. 6,171,576, which is incorporated herein in its entirety, e.g., (S)-(+)-2-carbomethoxy-3α-(bis(4-fluorophenyl)methoxy)tropane.

Additional examples of preferred boat tropane compounds are described in U.S. application Ser. No. 10/033,621 and include tropane analogs having the following formula:

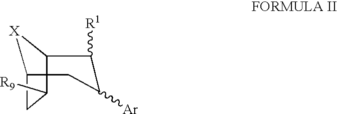

wherein:

R₁=COOR₇, COR_{3,} lower alkyl, lower alkenyl, lower alkynyl, CONHR₄, or COR₆ and is α or β;

R₉=OH or O, is a 6-or 7-substituent, and if R₉ is OH, it is α or β;

X=NR₃, CH₂, CHY, CYY₁, CO, O, S; SO, SO₂, NSO₂R₃, or C=CX₁Y with the N, C, O or S atom being a member of the ring;

X₁=NR₃, CH₂, CHY, CYY₁CO, O, S; SO, SO₂, or NSO₂R₃;

R₃=H, (CH₂)ₙC₆H₄Y, C₆H₄Y, CHCH₂, lower alkyl, lower alkenyl or lower alkynyl;

Y and Y₁=H, Br, Cl, I, F, OH, OCH₃, CF₃, NO₂, NH₂, CN, NHCOCH₃, N(CH₃)₂, (CH₂)nCH₃, COCH₃, or C(CH₃)₃;

R₄=CH₃, CH₂CH₃, or CH₃SO₂;

R₆=morpholinyl or piperidinyl;

Ar=phenyl-R₅, naphthyl-R₅, anthracenyl-R₅, phenanthrenyl-R₅, or diphenylmethoxy-R₅;

R₅=H, Br, Cl, I, F, OH, OCH₃, CF₃, NO₂, NH₂, CN, NHCOCH₃, N(CH₃)₂, (CH₂)nCH₃, COCH₃, C(CH₃)₃ where n=0-6, 4-F, 4-Cl, 4-I, 2-F, 2-Cl, 2-I, 3-F, 3-Cl, 3-I, 3,4-diCl, 3,4-diOH, 3,4-diOAc, 3,4-diOCH₃,3-OH-4-Cl, 3-OH-4-F, 3-Cl-4-OH, 3-F-4-OH, lower alkyl, lower alkoxy, lower alkenyl, lower alkynyl, CO(lower alkyl), or CO(lower alkoxy);

n=0, 1, 2, 3, 4 or 5;

R₇=lower alkyl; and

when X=N, R₁ is not COR₆.

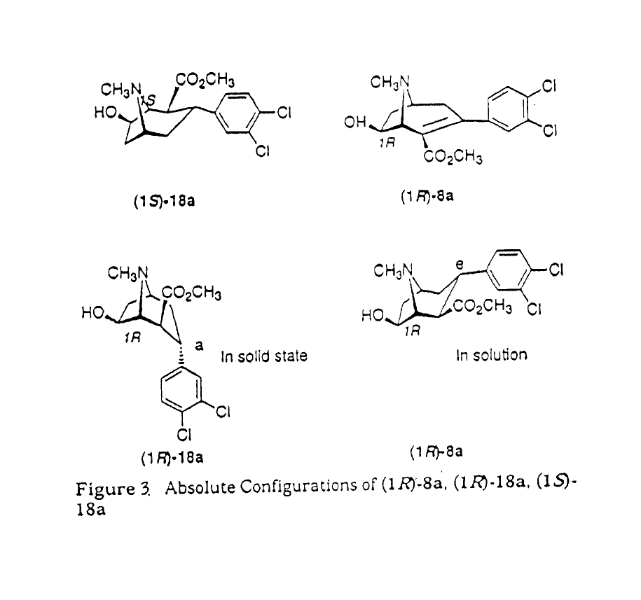

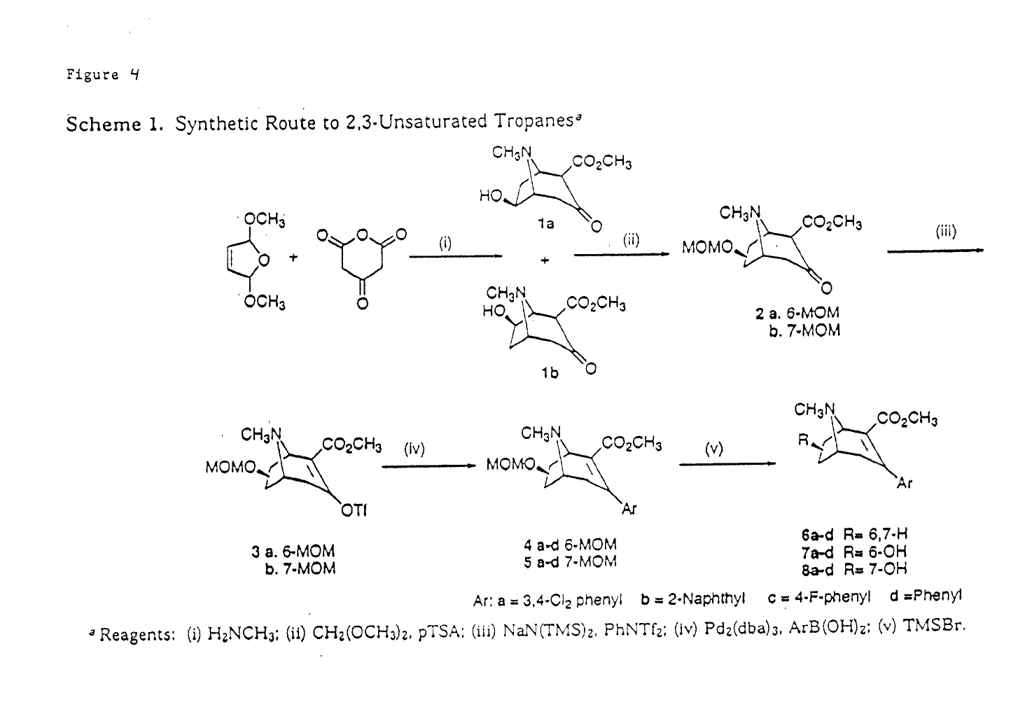

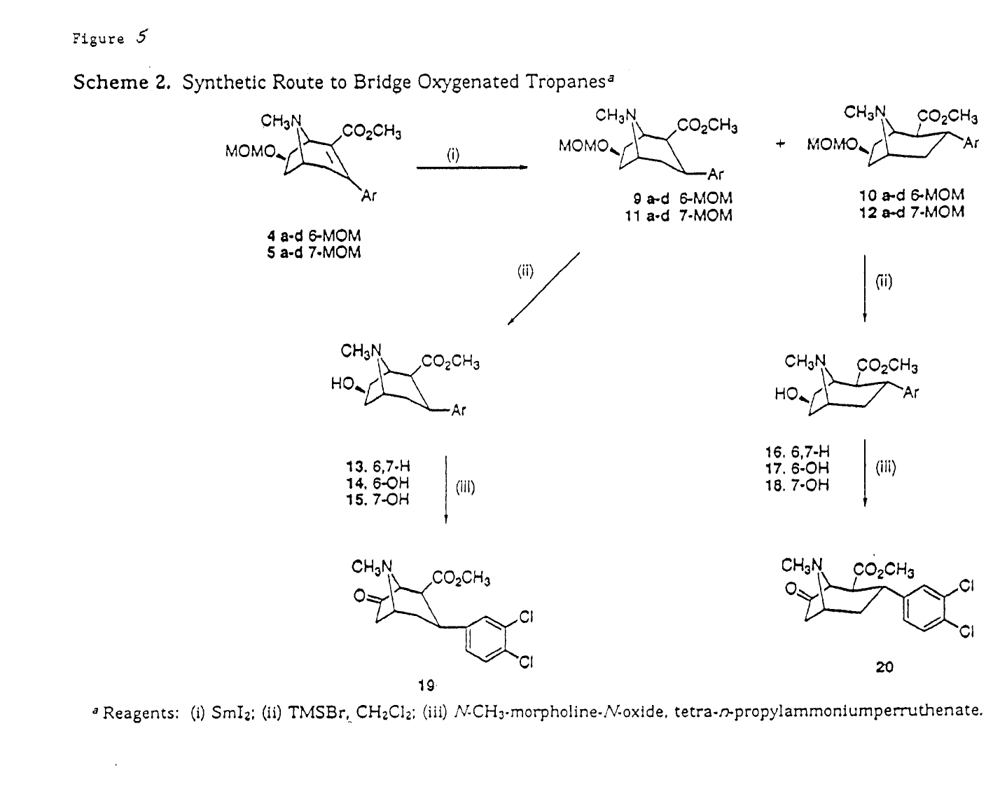

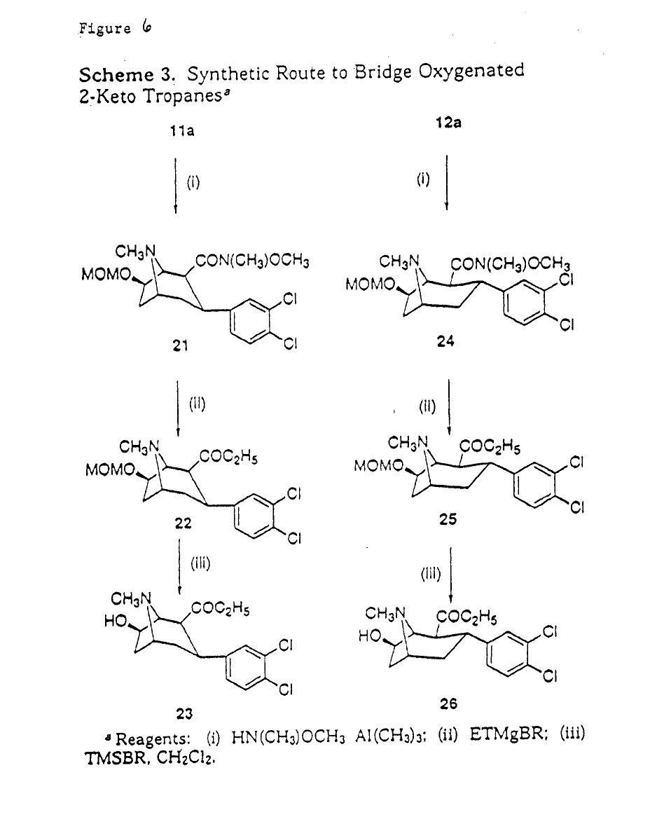

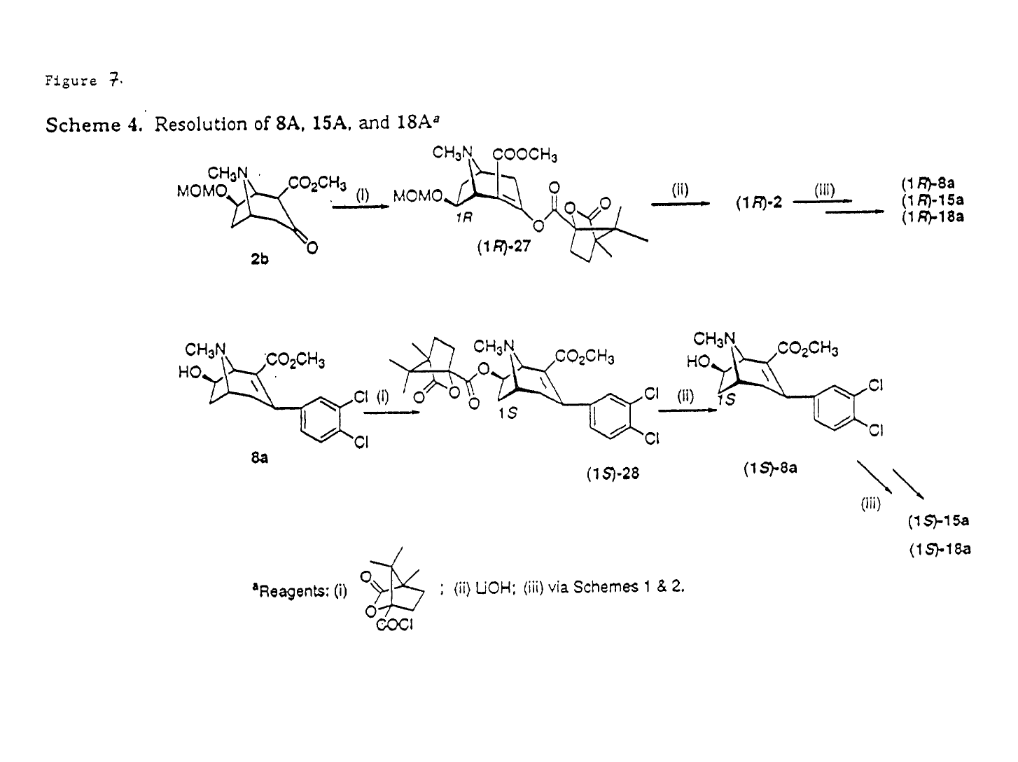

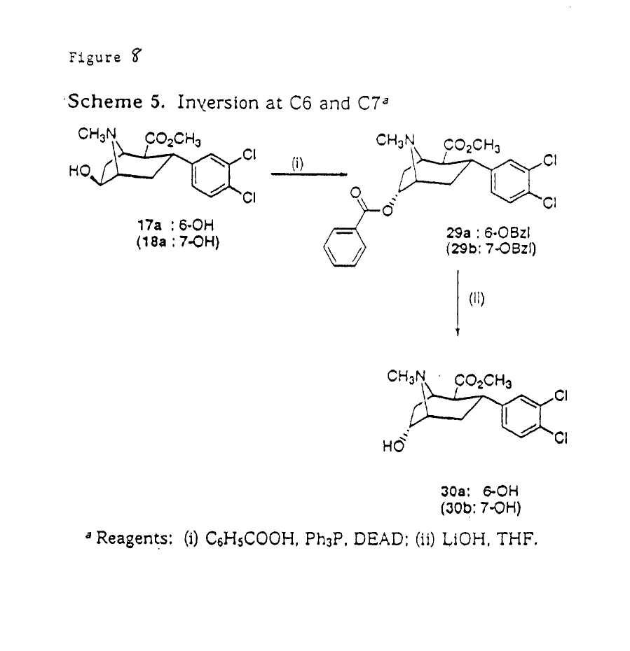

Synthetic routes to these compounds are shown in FIGS. 3-8 and described in U.S. application Ser. No. 10/033,621 and Meltzer, et al., J. Med. Chem. 2001, 44, 2619-2635.

The term “lower alkyl” when used herein designates aliphatic saturated branched or straight chain hydrocarbon monovalent substituents containing from 1 to about 8 carbon atoms such as methyl, ethyl, isopropyl, n-propyl, n-butyl, (CH₂)ₙCH₃, C(CH₃)₃; etc., more preferably 1 to 4 carbons. The term “lower alkoxy” designates lower alkoxy substituents containing from 1 to about 8 carbon atoms such as methoxy, ethoxy, isopropoxy, etc., more preferably 1 to 4 carbon atoms.

The term “lower alkenyl” when used herein designates aliphatic unsaturated branched or straight chain vinyl hydrocarbon substituents containing from 2 to about 8 carbon atoms such as allyl, etc., more preferably 2 to 4 carbons. The term “lower alkynyl” designates lower alkynyl substituents containing from 2 to about 8 carbon atoms, more preferably 2 to 4 carbon atoms such as, for example, propyne, butyne, etc.

The terms substituted lower alkyl, substituted lower alkoxy, substituted lower alkenyl and substituted lower alkynyl, when used herein, include corresponding alkyl, alkoxy, alkenyl or alkynyl groups substituted with halide, hydroxy, carboxylic acid, or carboxamide groups, etc. such as, for example, —CH₂OH, —CH₂CH₂COOH, —CH₂CONH₂, —OCH₂CH₂OH, —OCH₂COOH, —OCH₂CH₂CONH₂, etc. As used herein, the terms lower alkyl, lower alkoxy, lower alkenyl and lower alkynyl are meant to include where practical substituted such groups as described above.

When X contains a carbon atom as the ring member, reference to X is sometimes made herein as a carbon group. Thus, when X is a carbon group, as that phrase is used herein, it means that a carbon atom is a ring member at the X position (i.e., the 8-position).

The substituents at the 2 position of the ring can be α- or β. Preferred compounds have the substitutents at the 3-position in the α configuration to form the boat conformation. Although R₁ is illustrated in the 2-position, it should be recognized that substitution at the 4-position is also included and the position is dependent on the numbering of the tropane ring. The compounds of the present invention can be racemic, pure R-enantiomers, or pure S-enantiomers. Thus, the structural formulas illustrated herein are intended to represent each enantiomer and diastereomer of the illustrated compound. In certain preferred compounds of the present invention, R₁ is COOCH₃. In yet other preferred compounds, R₁ is COR₃, where R₃ is CHCH₂. Other preferred compounds are 6 or 7-bridge hydroxylated or keto compounds

Tropane analogs having hydroxyl or ketone substituents in the 6-or 7-position of the tropane structure include those having the formula:

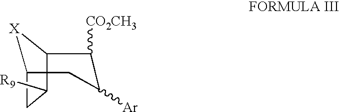

wherein X, Ar, and R₉ have the same meaning as defined above. In preferred compounds, R₉ is OH.

Preferred compounds for use in the present are those compounds wherein X is N, Ar is phenyl, substituted phenyl, diarylmethoxy or substituted diarylmethoxy. The aryl ring can be substituted with one or more halide atoms, hydroxy groups, nitro groups, amino groups, cyano groups, lower alkyl groups having from 1-8 carbon atoms, lower alkoxy groups having from 1-8 carbon atoms, lower alkenyl groups having from 2-8 carbon atoms, or lower alkynyl groups having from 2-8 carbon atoms. The aryl group can have a substituent selected from the group consisting of Br, Cl, I, F, OH, OCH₃, CF₃, NO₂, NH₂, CN, NHCOCH₃, N(CH₃)₂, COCH₃, C(CH₃)₃, (CH₂)ₙCH₃ where n=0-6, allyl, isopropyl and isobutyl. Preferably the substituent is a halogen. The aryl ring can be substituted with chloride, fluoride or iodide. Ar may be a mono- or di-halogen substituted phenyl. In certain embodiments, the amino group is a mono- or di-alkyl substituted group having from 1-8 carbon atoms. Examples of such compounds include, but are not limited to: 2β-Carbomethoxy-3α-(3,4-dichlorophenyl) -6β-hydroxy-8-methyl-8-azabicyclo{3.2.1}octane; 2β-Carbomethoxy-3α-(2-naphthyl)-6β-hydroxy-8-methyl-8-azabicyclo{3.2.1}octane; 2βCarbomethoxy-3α-(4-fluorophenyl)-6β-hydroxy-8-methyl-8-azabicyclo{3.2.1}octane; 2β-Carbomethoxy-3α-phenyl-6β-hydroxy-8-methyl-8-azabicyclo{3.2.1}octane; 2β-Carbomethoxy-3α(3,4-dichlorophenyl)-7β-hydroxy-8-methyl-8-azabicyclo{3.2.1}octane; (1S)-2β-Carbomethoxy-3α-(3,4-dichlorophenyl)-7β-hydroxy-8-methyl-8-azabicyclo{3.2.1}octane; (1R)-2β-Carbomethoxy-3α-(3,4-dichlorophenyl)-7β-hydroxy-8-methyl-8-azabicyclo{3.2.1}octane; 2β-Carbomethoxy-3α-(2-naphthyl)-7β-hydroxy-8-methyl-8-azabicyclo{3.2.1}octane; 2β-Carbomethoxy-3α-(4-fluorophenyl)-7β-hydroxy-8-methyl-8-azabicyclo{3.2.1}octane; 2β-Carbomethoxy-3α-phenyl-7β-hydroxy-8-methyl-8-azabicyclo{3.2.1}octane; 2β-Carbomethoxy-3α-(3,4-dichlorophenyl)-7α-benzoyloxy-8-methyl-8-azabicyclo{3.2.1}octane; 2β-Carbomethoxy-3α-(3,4-dichlorophenyl)-6α-benzoyloxy-8-methyl-8-azabicyclo{3.2.1}octane; 2β-Carbomethoxy-3α-(3,4-dichlorophenyl)-7α-hydroxy-8-methyl-8-azabicyclo{3.2.1}octane; 2β-Carbomethoxy-3α-(3,4-dichlorophenyl)-6α-hydroxy-8-methyl-8-azabicyclo{3.2.}octane; 2β-Carbomethoxy-3α-(3,4-dichlorophenyl)-8-methyl-8-azabicyclo{3.2.1}oct-7-one; 2β-Carbomethoxy-3β-(3,4-dichlorophenyl)-8-methyl-8-azabicyclo{3.2.1}oct-7-one; 2β-Carbomethoxy-3α-bis(fluorophenyl)methoxy-7β-hydroxy-8-methyl-8-azabicyclo{3.2.1}octane; and 2β-Carbomethoxy-3α-bis(4-fluorophenyl)methoxy-6β-hydroxy-8-methyl-8-azabicyclo{3.2.1 }octane.

Other preferred compounds have the following formula:

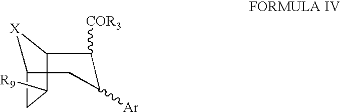

In preferred compounds, X includes a nitrogen, carbon or oxygen atom as a ring member, R₉ is OH, and Ar is phenyl, substituted phenyl such as mono- or di-halogen substituted phenyl, or a diarylmethoxy including halogen substituted such groups. In particularly preferred compounds, X is N₃, R₃ is CH₂CH₃, R₉ is OH or O in the 6- or 7-position, Ar is phenyl or naphthyl either of which can be substituted with halogen, alkenyl having 2-8 carbon atoms or alkynyl having 2-8 carbon atoms. Ar can be substituted with 4-Cl, 4-F, 4-Br, 4-I, 3,4-Cl₂, ethenyl, propenyl, butenyl, propynyl or butynyl.

In some preferred compounds, the compounds have a C2-ethylketone. One example of such a compound is 1-{3α-(3,4-Dichlorophenyl)-7β-hydroxy-8-methyl-8-azabicyclo{3.2.1}oct-2-yl}propan-1-one (Compound 26).

The bridge-hydroxylated tropane compounds provide a broad array of molecules including compounds that bind with very high affinity. Selectivity for inhibition of the DAT versus the serotonin transporter (SERT) is another property of tropanes of considerable relevance for development of medications and for probes useful to image the DAT in living brain. Preferred compounds for DAT imaging agents have high DAT:SERT selectivity.

Boat tropane compounds exhibit extremely potent and selective binding for the DAT. Compounds that have the desired target:non-target (DAT:SET) specificity can be selected based upon the particular use and application. Preferably, the selectivity ratio of binding of SERT to binding of DAT is greater than about 10, preferably greater than about 30 and more preferably 50 or more. In addition, preferred boat tropane compounds have an IC₅₀ less than about 500 nM, preferably less than 60 nM, more preferably less than about 20, and most preferably less than about 10. Using the combination of selectivity (SERT/DAT ratio) and potency (IC₅₀) information for these compounds, one of ordinary skill in the art can readily select the appropriate compound for the desired application, e.g., imaging or treatment.

Selectivity for inhibition of the DAT versus the SERT is greater for compounds bearing a 3α-aryl substituent as compared with a 3β-aryl substituent. Preferred compounds have the following substitutions at the C3 position: 3,4-dichlorophenyl, 2-naphthyl, 4-fluorophenyl, and phenyl.

Other preferred compounds for use in the methods of the present invention have a C2 ethyl ketone instead of a C2 ester. An especially preferred compounds a 3α-3,4-dichlorophenyl analog, with a C2 ethyl ketone, (compound 26). This compound is one of the most selective and potent DAT inhibitors (DAT: 1.1 nM; SERT: 2,520 nM) (see Scheme 3). Thus, in certain instances, preferred compounds for use in the present methods are substituted at the 20β-position, instead of a 2α-substitution. Other preferred compounds contain a C2-ketone, which retains potency at the DAT. Yet other preferred compounds are 6α- or 7α-hydroxylated compounds.

For use in the present invention, the compounds of interest can be made into pharmaceutical compositions, comprising the desired compounds in a pharmaceutically acceptable carrier. Pharmaceutically acceptable carriers are well known to those skilled in the art. An exemplary pharmaceutical composition is a therapeutically effective amount of a compound of the invention optionally included in a pharmaceutically-acceptable and compatible carrier. The term “pharmaceutically-acceptable and compatible carrier” as used herein, and described more fully below, refers to e.g., one or more compatible solid or liquid filler diluents or encapsulating substances that are suitable for administration to a human or other animal. The route of administration can be varied but is principally selected from intravenous, nasal and oral routes. For parenteral administration, e.g., it will typically be injected in a sterile aqueous or non-aqueous solution, suspension or emulsion in association with a pharmaceutically-acceptable parenteral carrier such as physiological saline.

The term “therapeutically-effective amount” is that amount of the pharmaceutical compositions which produces a desired result or exerts a desired influence on the particular condition being treated. Various concentrations may be used in preparing compositions incorporating the same ingredient to provide for variations in the age of the patient to be treated, the severity of the condition, the duration of the treatment and the mode of administration. An effective dose of the compound is administered to a patient based on IC₅₀ values determined in vitro.

The term “compatible”, as used herein, means that the components of the pharmaceutical compositions are capable of being commingled with the compounds of the present invention, and with each other, in a manner such that there is no interaction that would substantially impair the desired pharmaceutical efficacy.

Dose of the pharmaceutical compositions will vary depending on the subject and upon particular route of administration used. Pharmaceutical compositions of the present invention can also be administered to a subject according to a variety well-characterized protocols.

The pharmaceutical composition may a liquid composition in pyrogen-free, sterilized container or vial. The container can be unit dose or multidose.

The compounds and pharmaceutical preparations can be used to inhibit the %-hydroxytryptamine reuptake of a monoamine transporter, particularly reuptake by the dopamine transporter, serotonin transporter or norepinephrine transporter.

Dysfunction of dopamine neurons has been implicated in several neuropsychiatric diseases. Imaging of the dopamine neurons offers important clinical information relevant to diagnosis and therapeutic treatments. Dopamine neurons produce dopamine, release the neurotransmitter and remove the released dopamine with a dopamine transporter protein. Compounds that bind to the dopamine transporter are effective measures of dopamine neurons and can be transformed into imaging agents for PET and for SPECT imaging. In identifying a suitable compound for the dopamine transporter, an essential first step is to measure the affinity and selectivity of a candidate at the dopamine transporter. The affinity is measured by conducting radioreceptor assays. A radiolabeled marker for the transporter, e.g., (³H)WIN 35,428, is incubated with the unlabeled candidate and a source of the transporter, usually brain striatum. The effect of various concentrations of the candidate on inhibiting (3H)WIN 35,428 binding is quantified. The concentration of the compound that inhibits 50% of (³H)WIN 35,428 bound to the transporter (IC₅₀ value) is used as a measure of its affinity for the transporter. A suitable range of concentrations of the candidate typically is 1-10 nM.

It is also important to measure the selectivity of the candidate of the dopamine compared with the serotonin transporter. The serotonin transporter is also detectable in the striatum, the brain region with the highest density of dopamine neurons and in brain regions surrounding the striatum. It is necessary to determine whether the candidate compound is more potent at the dopamine than the serotonin transporter. If more selective (>10-fold), the probe will permit accurate measures of the dopamine transporter in this region of interest or will provide effective treatment modality for the dopamine transporter. Therefore, a measure of probe affinity of the serotonin transport is conducted by assays paralleling the dopamine transporter assays. (³H)Citalopram is used to radiolabel binding sites on the serotonin transporter and competition studies are conducted with the candidate compound at various concentrations in order to generate an IC₅₀ value.

This invention will be illustrated further by the following examples. These examples are not intended to limit the scope of the claimed invention in any manner. The Examples provide suitable methods for preparing compounds of the present invention. However, those skilled in the art may make compounds of the present invention by any other suitable means. As is well known to those skilled in the art, other substituents can be provided for the illustrated compounds by suitable modification of the reactants.

All exemplified target compounds are fully analyzed (mp, TLC, CHN, GC and/or HPLC) and characterized (¹H NMR, ¹³C NMR, MS, IR) prior to submission for biological evaluation. The affinity of all the compounds for the DAT, SERT and NET are measured. NMR spectra are recorded on a Bruker 100, a Varian XL 400, or a Bruker 300 NMR spectrometer. Tetramethylsilane (“TMS”) is used as internal standard. Melting points are uncorrected and are measured on a Gallenkamp melting point apparatus. Thin layer chromatography (TLC) is carried out on Baker Si 250F plates. Visualization is accomplished with iodine vapor, UV exposure or treatment with phosphomolybdic acid (PMA). Preparative TLC is carried out on Analtech uniplates Silica Gel GF 2000 microns. Flash chromatography is carried out on Baker Silica Gel 40 mM. Elemental Analyses are performed by Atlantic Microlab, Atlanta, Ga. and are within 0.4% of calculated values for each element. A Beckman 1801 Scintillation Counter is used for scintillation spectrometry. 0.1% Bovine Serum Albumin (“BSA”) and (−)-cocaine is purchased from Sigma Chemicals. All reactions are conducted under an inert (N₂) atmosphere.

³H-WIN 35,428 (³H-CFT, 2β-carbomethoxy-3β-(4-fluorophenyl)-N-³H-methyltropane, 79.4-87.0 Ci/mmol) and ³H-citalopram (86.8 Ci/mmol) is purchased from DuPont-New England Nuclear (Boston, Mass.). (R)-(−)-Cocaine hydrochloride for the pharmacological studies was donated by the National Institute on Drug Abuse (NIDA). Fluoxetine was donated by E. Lilly & Co. HPLC analyses are carried out on a Waters 510 system with detection at 254 nm on a Chiralcel OC column (flow rate: 1 mL/min).

Preparation of Compounds

Reaction schemes for preparation of various classes of compounds of the present invention are described with reference to the drawings. In Scheme 1, as illustrated in FIG. 1, Keto ester 1′{Meltzer et al., J. Med. Chem, 1994, 37, 2001} is converted to the enol triflate 2′ by reaction with N-phenyltrifluoromethanesulfonimide and sodium bis(trimethylsilyl)amide in tetrahydrofuran. The enol triflate 2′ is then coupled with the appropriate commercial or preformed arylboronic acids by Suzuki coupling in diethoxymethane in the presence of lithium chloride, sodium carbonate and tris(dibenzylideneacetone)dipalladium(0) to provide aryl octenes 3′ in excellent yield.

Reduction of the octenes 3′ with samarium iodide in tetrahydrofuran/methanol at low temperature (−78° C.) provides a mixture of the 3β- and 3α-diastereomers, 4′ and 12′ respectively. These diastereomers are readily separated by flash column chromatography.

### EXAMPLE 1

### 2β-(1-Propanoyl)-3α-(4-fluorophenyl)-tropane (Compound 15′ (R=4-F), FIG. 2)

A 250 mL round bottom flask containing the 2β-methoxymethylcarbamoyl-3α-(4-fluorophenyl)tropane 14′ (471 mg) was flushed with nitrogen and charged with anhydrous THF (70 mL). At room temperature, EtMgBr/Et₂O (3.0 mL; 3.0M) was added dropwise over 3 min. The reaction was stirred at room temperature for 30 min and was then heated to 65° C. for 1h at which point no starting material was observed by TLC (TLC sample was prepared by adding an aliquot of the reaction to ethereal HCl, and basifying with 2M Na₂CO_{3;} R_{f} (product) 0.42; R_{f} (starting material) 0.13 (20% EtOAc/hexanes, 5% Et₃N). The reaction was cooled in an ice bath and quenched by slow addition of ethereal HCl. The cloudy solution was basified with 2M Na₂CO₃ and diluted with ether (25 mL). The layers were separated and the aqueous layer was extracted with ether (1×10 mL) and CHCl₃ (2×20 mL). The combined organic extracts were dried (Na₂SO₄), filtered and reduced in vacuo to yield the crude residue (484 mg). This residue was then chromatographed (25 g SiO₂; eluent 25% EtOAc/hexanes, 5% Et₃N). Fractions containing the product were combined and concentrated to yield 15′ (300 mg, 70%).

Mp. 60.5-61.3° C.; R_{f} 0.49 (33% EtOAc/hexanes; 5% Et₃N); ¹H-NMR (CDCl₃) δ0.86 (t, 3H), 1.27 (ddd, 1H), 1.4-1.6 (m, 2H), 2.0-2.5 (m, 6H), 2.23 (s, 3H), 3.12 (brd, 1H), 3.2-3.3 (m, 2H), 6.85-7.0 (m, 2H), 7.05-7.15 (m, 2H); IR (KBr) 2900, 1740, 1500 cm⁻¹; Elemental analysis: calculated C, 74.15; H, 8.05; N, 5.09; found C, 74.00; H, 8.13; N, 4.98.

### EXAMPLE 2

### 2β-(1-Propanoyl)-3α-(3,4-dichlorophenyl)tropane (Compound 15′ (R=3,4-Cl₂), FIG. 2)

2β-Methoxymethylcarbamoyl-3α-(3,4-dichlorophenyl)tropane, 14′ (105 mg, 0.29 mmol) was flushed with nitrogen and charged with anhydrous THF (15 mL). At room temperature, EtMgBr/Et₂O (0.8 mL; 3.0M) was added dropwise over 3 min. The reaction was stirred at room temperature for 1h and was then heated to 55° C. for 30 min at which point no starting material was observed by TLC. The reaction was cooled in an ice bath and quenched by slow addition of ethereal HCl. The cloudy solution was basified with 2M Na₂CO₃ and diluted with ether (15 mL) and water (15 mL). The layers were separated and the aqueous layer was extracted with CHCl₃ (2×15 mL). The combined organic extracts were dried (Na₂SO₄), filtered and reduced in vacuo to yield a residue (95 mg) which was chromatographed (5 g SiO₂, eluent 25% EtOAc in hexanes, 5% Et₃N). Fractions containing the product were combined and concentrated to yield 15′ (80 mg, 80%).

R_{f} 0.28 (30% EtOAc/hexanes; 5% Et₃N); ¹H-NMR (CDCl₃) δ0.93 (t, J=7.4 Hz, 3H), 1.27 (ddd, 1H), 1.42-1.62 (m, 2H), 2.06-2.30 (m, 6H), 2.21 (s, 3H), 3.32 2.52 (m, 3H), 3.14 (brd 1H), 3.2-3.36 (m, 2H), 7.10 (dd, 1H), 7.24 (d, 1H), 7.29 (d, 1H).

### EXAMPLE 2a

### 2β-(1-Propanoyl)-3α-(3,4-dichlorophenyl)tropane (Compound 15′ (R-3,4-Cl₂), FIG. 2)

To commercially available ethylmagnesium bromide (1M in THF, 12.6 mL, 12.6 mmol) in a flask equipped with an addition funnel under nitrogen was added triethylamine (5.0 g, 50.4 mmol). To the resulting mixture was added drop-wise a solution of compound 12′ (R=Cl_{2,} 750 mg, 2.29 mmol) in benzene (10 mL) at 5-10° C. over a period of 1 hour. The reaction mixture was then stirred at 5-10° C. for 5 hours and then treated with 4 M HCl (2.9 mL, 11.6 mmol). The organic layer was washed with water (1×50 mL), 5% NaHCO₃ (aq) (1×50 mL) and water (2×50 mL). The organic phase was then dried (K₂CO₃), filtered and the concentrated. The residue was chromatographed (SiO₂. 25% EtOAc in hexanes with 5% Et₃N) and gave 670 mg (85%) of compound 15′ with the same physical and spectral characteristics as previously reported (Example 1).

### EXAMPLE 3

### Tissue Sources and Preparation

Brain tissue from adult male and female cynomolgus monkeys (Macaca fasicularis) and rhesus monkeys (Macaca mulatta) was stored at −85° C. in the primate brain bank at the New England Regional Primate Research Center. We recently cloned the DAT and SERT from both species and found them to have virtually identical protein sequences (Miller, G. M. et al., Brain Res. Mol. Brain Res. 2001, 87, 124-143). The caudate-putamen was dissected from coronal slices and yielded 1.4±0.4 g tissue. Membranes were prepared as described previously. Briefly, the caudate-putamen was homogenized in 10 volumes (w/v) of ice-cold Tris.HCl buffer (50 mM, pH 7.4 at 4° C.) and centrifuged at 38,000×g for 20 min in the cold. The resulting pellet was suspended in 40 volumes of buffer, and the entire was procedure was repeated twice. The membrane suspension (25 mg original wet weight of tissue/ml) was diluted to 12 ml/ml for {³H}WIN 35,428 or {³H}citalopram assay in buffer just before assay and was dispersed with a Brinkmann Polytron homogenizer (setting #5) for 15 sec. All experiments were conducted in triplicate and each experiment was repeated in each of 2-3 preparations from individual brains.

### EXAMPLE 4

### Dopamine Transporter Assay

The dopamine transporter was labeled with {³H}WIN 35,428 ({³H}CFT, (1 R)-2β-carbomethoxy-3β-(4-fluorophenyl)-N-{³H}methyltropane, 81-84 Ci/mmol, DuPont-NEN). The affinity of {³H}WIN 35,428 for the dopamine transporter was determined in experiments by incubating tissue with a fixed concentration of {³H}WIN 35,428 and a range of concentration of unlabeled WIN 35,428. The assay tubes received, in Tris.HCl buffer (50 mM, pH 7.4 at 0-4° C.; NaCl 100 mM), the following constituents at a final assay concentration: WIN35,428, 0.2 ml (1 pM -100 or 300 nM), {³H}WIN 35,428 (0.3 nM); membrane preparation 0.2 mL (4 mg original wet weight of tissue/mL). The 2 h incubation (0-4° C.) was initiated by addition of membranes and terminated by rapid filtration over Whatman GF/B glass fiber filters pre-soaked in 0.1% bovine serum albumin (Sigma Chem. Co.). The filters were washed twice with 5 mL Tris.HCl buffer (50 mM), incubated overnight at 0-4 ° C. in scintillation fluor (Beckman Ready-Value, 5 mL) and radioactivity was measured by liquid scintillation spectrometry (Beckman 1801). Cpm were converted to dpm following determination of counting efficiency (>45%) of each vial by external standardization.

Total binding was defined as {³H}WIN 35,428 bound in the presence of ineffective concentrations of unlabeled WIN 35,428 (1 or 10 pM). Non-specific binding was defined as {³H}WIN 35,428 bound in the presence of an excess (30 μM) of (−)-cocaine. Specific binding was the difference between the two values. Competition experiments to determine the affinities of other drugs at {³H}WIN 35,428 binding sites were conducted using procedures similar to those outlined above. Stock solutions of water-soluble drugs were dissolved in water or buffer and stock solutions of other drugs were made in a range of ethanol/HCl solutions or other appropriate solvents. Several of the drugs were sonicated to promote solubility. The stock solutions were diluted serially in the assay buffer and added (0.2 mL) to the assay medium as described above. IC₅₀ values were computed by the EBDA computer program and are the means of experiments conducted in triplicate.

### EXAMPLE 5

### Serotonin Transporter Assay

The serotonin transporter was assayed in caudate-putamen membranes using conditions similar to those for the dopamine transporter. The affinity of {³H}citalopram (spec. act.: 82 Ci/mmol, DuPont-NEN) for the serotonin transporter was determined in experiments by incubating tissue with a fixed concentration of {³H}citalopram and a range of concentrations of unlabeled citalopram. The assay tubes received, in Tris.HCl buffer (50 mM, pH 7.4 at 0-4° C.; NaCl 100 mM), the following constituents at a final assay concentration: citalopram, 0.2 ml (1 pM-100 or 300 nM), {³H}citalopram (1 nM); membrane preparation 0.2 ml (4 mg original wet weight of tissue/mL). The 2 h incubation (0-4° C.) was initiated by addition of membranes and terminated by rapid filtration over Whatman GF/B glass fiber filters pre-soaked in 0.1% polyethyleneimine. The filters were washed twice with 5 ml Tris.HCl buffer (50 mM), incubated overnight at 0-4° C. in scintillation fluor (Beckman Ready-Value, 5 mL) and radioactivity was measured by liquid scintillation spectrometry (Beckman 1801). Cpm were converted to dpm following determination of counting efficiency (>45%) of each vial by external standardization. Total binding was defined as {³H}citalopram bound in the presence of ineffective concentrations of unlabeled citalopram (1 or 10 pM). Non-specific binding was defined as {³H}citalopram bound in the presence of an excess (10 pM) of fluoxetine. Specific binding was the difference between the two values. Competition experiments to determine the affinities of other drugs at {³H}citalopram binding sites were conducted using procedures similar to those outlined above. IC₅₀ values were computed by the EBDA computer program and are the means of experiments conducted in triplicate.

Table 1 presents binding data for the 7-keto, 6α- and 7α-hydroxy, and 3-diarylmethoxy tropane compounds shown in FIGS. 3-8. Table 1 shows the inhibition of {³H}WIN 35,428 binding to the dopamine transporter and {³H}citalopram binding to the serotonin transporter in rhesus or cynomolgus monkey caudate-putamen. Studies were conducted in monkey striatum because this tissue (Meltzer, P. C. et al., Med. Chem. Res. 1998, 8, 12-34) is used in an ongoing investigation of structure activity relationships at the DAT, and meaningful comparisons with an extensive database can be made. Competition studies were conducted with a fixed concentration of radioligand and a range of concentrations of the test drug. All drugs inhibited {³H}WIN 35,428 and {³H}citalopram binding in a concentration-dependent manner. Each value is the mean of 2 or more independent experiments each conducted in different brains and triplicate. Errors generally do not exceed 15% between replicate experiments. Highest doses tested were generally 10-100 μM.

TABLE 1

IC₅₀ (nM)

| | | |
|---|---|---|
| Compound | DAT | SERT |

| | | | |
|---|---|---|---|
| 20 | O-2096 | 14.2 | 7,038 |
| 26 | O-2099 | 1.1 | 2,520 |
| 30a | O-2015 | 33.2 | 10,700 |
| 30b | O-2032 | 3.04 | 991 |

Table 2 presents a comparison of 6- and 7-hydroxylated compounds as well as bridge unsubstituted (R₉=H) parent compounds.

TABLE 2

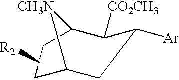

Ar: a = 3,4-Cl₂ phenyl
b = 2-naphthyl
c = 4-F-phenyl
d = phenyl

| | | IC₅₀ (nM) | | |
|---|---|---|---|---|
| R₂ | Compound | DAT | SERT | SERT/DAT |
| H | 16a, O-1157 (R) | 0.38 | 27.7 | 73 |
| 6-OH | 17a, O-1926 | 6.09 | 1,450 | 238 |
| 7-OH | 18a, O-1163 | 1.19 | 1,390 | 1,170 |
| 7-OH | (1R)-18a, O-1676 | 482 | 5,300 | 11 |
| 7-OH | (1S)-18a, O-1924 | 0.76 | 1,220 | 1,610 |
| H | 16b, O-1228 | 057 | 5.95 | 10 |
| 6-OH | 17b, O-1748 | 32 | 180 | 6 |
| 7-OH | 18b, O-1952 | 28 | 94 | 34 |
| H | 16c, O-1204 | 17.9 | 1,130 | 63 |
| 6-OH | 17c, O-1755 | 739 | 5,820 | 8 |
| 7-OH | 18c, O-1951 | 110 | >20,000 | >120 |
| H | 16d | NA | NA | — |
| 6-OH | 17d, O-1589 | 3,530 | >10,000 | >3 |
| 7-OH | 18d, O-1954 | 518 | >100,000 | >190 |

In general, the 7-hydroxy compounds (18) are more potent than the 6-hydroxy compounds (17). When the aromatic ring is oriented in the 3α-configuration, 10 the parent-unsubstituted compound (1R)-16a has DAT IC₅₀ =0.38 nM and the hydroxylated enantiopure compound (1S)-18a shows a similar value of 0.76 nM. In this case, the hydroxylated compound shows a selectivity ratio of 1610 and is therefore 22-fold more selective than 16a. However, the (1S)-18a, the 3α-configured compounds, is 32-fold more selective than its 3β-counterpart (data not shown). Thus, introduction of an hydroxyl at C7 has, at least, maintained potency of DAT inhibition and retained or may have increased selectivity versus inhibition of the SERT. This increase in selectivity is evident in the 6-hydroxy compounds (17a) as well.

The present invention has been described in detail, including the preferred embodiments thereof. However, it will be appreciated that those skilled in the art, upon consideration of the present disclosure, may make modifications and/or improvements of this invention and still be within the scope and spirit of this invention as set forth in the following claims.

All references cited are incorporated herein in their entirety by reference.

We claim:

1. A method of treating a neurological disorder in a patient comprising administering to the patient an effective amount of a boat tropane compound.

2. The method according to claim 1, wherein the compound has the following structure: 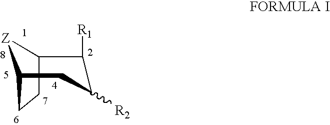
wherein
R₁ is α or β and is selected from COOR^{a}, COR^{a}, and CON(CH₃)OR^{a};
R₂ is α and is selected from C₆H₄X, C₆H₃XY, C₁₀H₇X, and C₁₀H₆XY;
R^{a} is selected from C₁-C₅ alkyl, e.g. methyl, ethyl, propyl, isopropyl;
X and Y are independently selected from R^{a}, H, Br, Cl, I, F, OH, and OCH₃,
Z=NR₃, NSO₂R₃, with R₃=H, (CH₂)ₙC₆H₄Y, C₆H₄Y, CHCH₂, lower alkyl, lower alkenyl or lower alkynyl.

3. The method according to claim 1, wherein the compound comprises 2β-(1-Propanoyl)-3α-(4-fluorophenyl)-tropane.

4. The method according to claim 1, wherein the compound comprises 2β-(1-Propanoyl)-3α-(3,4-dichlorophenyl)tropane.

5. The method according to claim 1, wherein the compound has the following structure: 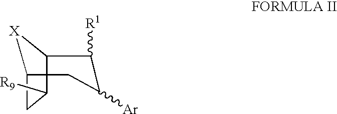
wherein:
R₁=COOR₇, COR₃, lower alkyl, lower alkenyl, lower alkynyl, CONHR₄, or COR₆ and is α or β;
R₉=OH or O, is a 6- or 7-substituent, and if R₉ is OH, it is α or β;
X=NR₃, CH₂, CHY, CYY₁, CO, O, S; SO, SO₂, NSO₂R₃, or C=CX₁Y with the N, C, O or S atom being a member of the ring;
X₁=NR₃, CH₂, CHY, CYY₁ CO, O, S; SO, SO₂, or NSO₂R₃;
R₃=H, (CH₂)ₙC₆H₄Y, C₆H₄Y, CHCH₂, lower alkyl, lower alkenyl or lower alkynyl;
Y and Y₁=H, Br, Cl, I, F, OH, OCH₃, CF₃, NO₂, NH₂, CN, NHCOCH₃, N(CH₃)₂, (CH₂)nCH₃, COCH₃, or C(CH₃)₃;
R₄=CH₃, CH₂CH₃, or CH₃SO₂;
R₆=morpholinyl or piperidinyl;
Ar=phenyl-R₅, naphthyl-R₅, anthracenyl-R₅, phenanthrenyl-R₅, or diphenylmethoxy-R₅;
R₅=H, Br, Cl, I, F, OH, OCH₃, CF₃, NO₂, NH₂, CN, NHCOCH₃, N(CH₃)₂, (CH₂)nCH₃, COCH₃, C(CH₃)₃ where n=0-6, 4-F, 4-Cl, 4-I, 2-F, 2-Cl, 2-I 3-F, 3-Cl, 3-I, 3,4-diCl, 3,4-diOH, 3,4-diOAc, 3,4-diOCH₃, 3-OH-4-Cl, 3-OH-4-F, 3-Cl-4-OH, 3-F-4-OH, lower alkyl, lower alkoxy, lower alkenyl, lower alknyl, CO(lower alkyl), or CO(lower alkoxy);
n=0, 1, 2, 3, 4 or 5;
R₇=lower alkyl; and
when X=N, R₁ is not COR₆.

6. The method according to claim 5, wherein R₁=CO₂CH₃.

7. The method according to claim 1, wherein the compound is selected from the following compounds:
2β-Carbomethoxy-3α-(3,4-dichlorophenyl) -6β-hydroxy-8-methyl-8-azabicyclo{3.2.1}octane;
2β-Carbomethoxy-3α-(2-naphthyl)-6β-hydroxy-8-methyl-8-azabicyclo{3.2.1.}octane;
2β-Carbomethoxy-3α-(4-fluorophenyl)-6β-hydroxy-8-methyl-8-azabicyclo{3.2.1}octane;
2β-Carbomethoxy-3α-phenyl-6β-hydroxy-8-methyl-8-azabicyclo{3.2.1}octane;
2β-Carbomethoxy-3α-(3,4-dichlorophenyl)-7β-hydroxy-8-methyl-8-azabicyclo{3.2.1}octane;
(1S)-2β-Carbomethoxy-3α-(3,4-dichlorophenyl)-7β-hydroxy-8-methyl-8-azabicyclo{3.2.1}octane;
(1R)-2β-Carbomethoxy-3α-(3,4-dichlorophenyl)-7β-hydroxy-8-methyl-8-azabicyclo{3.2.1}octane;
2β-Carbomethoxy-3α-(2-naphthyl)-7β-hydroxy-8-methyl-8-azabicyclo{3.2.1}octane;
2β-Carbomethoxy-3α-(4-fluorophenyl)-7β-hydroxy-8-methyl-8-azabicyclo{3.2.1}octane;
2β-Carbomethoxy-3α-phenyl-7β-hydroxy-8 -methyl-8-azabicyclo{3.2.1}octane;
2β-Carbomethoxy-3α-(3,4-dichlorophenyl)-7α-benzoyloxy-8-methyl-8-azabicyclo{3.2.1}octane;
2β-Carbomethoxy-3α-(3,4-dichlorophenyl)-6α-benzoyloxy-8-methyl-8-azabicyclo{3.2.1}octane;
2β-Carbomethoxy-3α-(3,4-dichlorophenyl)-7α-hydroxy-8-methyl-8-azabicyclo{3.2.1}octane;
2β-Carbomethoxy-3α-(3,4-dichlorophenyl)-6α-hydroxy-8-methyl-8-azabicyclo{3.2.1}octane;
2β-Carbomethoxy-3α-(3,4-dichlorophenyl)-8-methyl-8-azabicyclo{3.2.1}oct-7-one;
2β-Carbomethoxy-3β-(3,4-dichlorophenyl)-8-methyl-8-azabicyclo{3.2.1}oct-7-one;
2β-Carbomethoxy-3α-bis(fluorophenyl)methoxy-7β-hydroxy-8-methyl-8-azabicyclo{3.2.1}octane; and
2β-Carbomethoxy-3α-bis(4-fluorophenyl)methoxy-6β-hydroxy-8-methyl-8-azabicyclo{3.2.1}octane.

8. The method according to claim 5, wherein R₁=COR₃.

9. The method according to claim 1, wherein the compound comprises 1{3α-(3,4-Dichlorophenyl)-7β-hydroxy-8-methyl-8-azabicyclo{3.2.1}oct-2-yl}propan-1-one.

10. The method according to claim 1, wherein the neurological disorder is selected from a neurodegenerative disease, dopamine dysfunction, psychiatric dysfunction, and clinical dysfunction.

11. A method for inhibiting 5-hydroxytryptamine reuptake of a monoamine transporter comprising contacting the monoamine transporter with a boat tropane compound.

12. The method of claim 11, wherein the monoamine transporter is selected from the group consisting of a dopamine transporter, a serotonin transporter and a norepinephrine transporter.

13. A method of treating a neurodegenerative disease in a patient comprising administering to the patient an effective amount of a boat tropane compound selected from the following compounds:
2β-(1-Propanoyl)-3α-(4-fluorophenyl)-tropane;
2β-(1-Propanoyl)-3α-(3,4-dichlorophenyl)tropane;
1-{3α-(3,4-Dichlorophenyl)-7β-hydroxy-8-methyl-8-azabicyclo{3.2.1}oct-2-yl}propan-1-one;
2β-Carbomethoxy-3α-(3,4-dichlorophenyl)-6β-hydroxy-8-methyl-8-azabicyclo{3.2.1}octane;
2β-Carbomethoxy-3α-(2-naphthyl)-6β-hydroxy-8-methyl-8-azabicyclo{3.2.1}octane;
2β-Carbomethoxy-3α-(4-fluorophenyl)-6β-hydroxy-8-methyl-8-azabicyclo{3.2.1}octane;
2β-Carbomethoxy-3α-phenyl-6β-hydroxy-8-methyl-8-azabicyclo{3.2.1}octane;
2β-Carbomethoxy-3α-(3,4-dichlorophenyl)-7β-hydroxy-8-methyl-8-azabicyclo{3.2.1}octane;
(1S)-2β-Carbomethoxy-3α-(3,4-dichlorophenyl)-7β-hydroxy-8-methyl-8-azabicyclo{3.2.1}octane;
(1R)-2β-Carbomethoxy-3α-(3,4-dichlorophenyl)-7β-hydroxy-8-methyl-8-azabicyclo{3.2.1}octane;
2β-Carbomethoxy-3α-(2-naphthyl)-7β-hydroxy-8-methyl-8-azabicyclo{3.2.1}octane;
2β-Carbomethoxy-3α-(4-fluorophenyl)-7β-hydroxy-8-methyl-8-azabicyclo{3.2.1}octane;
2β-Carbomethoxy-3α-phenyl-7β-hydroxy-8-methyl-8-azabicyclo{3.2.1}octane;
2β-Carbomethoxy-3α-(3,4-dichlorophenyl)-7α-benzoyloxy-8-methyl-8-azabicyclo{3.2.1}octane;
2β-Carbomethoxy-3α-(3,4-dichlorophenyl)-6α-benzoyloxy-8-methyl-8-azabicyclo{3.2.1}octane;
2β-Carbomethoxy-3α-(3,4-dichlorophenyl)-7α-hydroxy-8-methyl-8-azabicyclo{3.2.1}octane;
2β-Carbomethoxy-3α-(3,4-dichlorophenyl)-6α-hydroxy-8-methyl-8-azabicyclo{3.2.1}octane;
2β-Carbomethoxy-3α-(3,4-dichlorophenyl)-8-methyl-8-azabicyclo{3.2.1}oct-7-one;
2β-Carbomethoxy-3β-(3,4-dichlorophenyl)-8-methyl-8-azabicyclo{3.2.1}oct-7-one;
2β-Carbomethoxy-3α-bis(fluorophenyl)methoxy-7β-hydroxy-8-methyl-8-azabicyclo{3.2.1}octane; and
2β-Carbomethoxy-3α-bis(4-fluorophenyl)methoxy-6β-hydroxy-8-methyl-8-azabicyclo{3.2.1}octane.

14. The method of claim 13, wherein the neurodegenerative disease is selected from Parkinson's disease and Alzheimer's disease.

15. A method for treating psychiatric dysfunction in a mammal comprising administering to the mammal an effective amount of a boat tropane compound selected from the following compounds:
2β-(1-Propanoyl)-3α-(4-fluorophenyl)-tropane;
2β-(1-Propanoyl)-3α-(3,4-dichlorophenyl)tropane;
1-{3α-(3,4-Dichlorophenyl)-7β-hydroxy-8-methyl-8-azabicyclo{3.2.1}oct-2-yl}propan-1-one;
2β-Carbomethoxy-3α-(3,4-dichlorophenyl)-6β-hydroxy-8-methyl-8-azabicyclo{3.2.1}octane;
2β-Carbomethoxy-3α-(2-naphthyl)-6β-hydroxy-8-methyl-8-azabicyclo{3.2.1}octane;
2β-Carbomethoxy-3α-(4-fluorophenyl)-6β-hydroxy-8-methyl-8-azabicyclo{3.2.1}octane;
2β-Carbomethoxy-3α-phenyl-6β-hydroxy-8-methyl-8-azabicyclo{3.2.1}octane;
2β-Carbomethoxy-3α-(3,4-dichlorophenyl)-7β-hydroxy-8-methyl-8-azabicyclo{3.2.1}octane;
(1S)-2β-Carbomethoxy-3β-(3,4-dichlorophenyl)-7β-hydroxy-8-methyl-8-azabicyclo{3.2.1}octane;
(1R)-2β-Carbomethoxy-3α-(3,4-dichlorophenyl)-7β-hydroxy-8-methyl-8-azabicyclo{3.2.1}octane;
2β-Carbomethoxy-3α-(2-naphthyl)-7β-hydroxy-8-methyl-8-azabicyclo{3.2.1}octane;
2β-Carbomethoxy-3α-(4-fluorophenyl)-7β-hydroxy-8-methyl-8-azabicyclo{3.2.1}octane;
2β-Carbomethoxy-3α-phenyl-7β-hydroxy-8-methyl-8-azabicyclo{3.2.1}octane;
2β-Carbomethoxy-3α-(3,4-dichlorophenyl)-7α-benzoyloxy-8-methyl-8-azabicyclo{3.2.1}octane;
2β-Carbomethoxy-3α-(3,4-dichlorophenyl)-6α-benzoyloxy-8-methyl-8-azabicyclo{3.2.1}octane;
2β-Carbomethoxy-3α-(3,4-dichlorophenyl)-7α-hydroxy-8-methyl-8-azabicyclo{3.2.1}octane;
2β-Carbomethoxy-3α-(3,4-dichlorophenyl)-6α-hydroxy-8-methyl-8-azabicyclo{3.2.1}octane;
2β-Carbomethoxy-3α-(3,4-dichlorophenyl)-8-methyl-8-azabicyclo{3.2.1}oct-7-one;
2β-Carbomethoxy-3β-(3,4-dichlorophenyl)-8-methyl-8-azabicyclo{3.2.1}oct-7-one;
2β-Carbomethoxy-3α-bis(fluorophenyl)methoxy-7β-hydroxy-8-methyl-8-azabicyclo{3.2.1}octane; and
2β-Carbomethoxy-3α-bis(4-fluorophenyl)methoxy-6β-hydroxy-8-methyl-8-azabicyclo{3.2.1}octane.

16. The method according to claim 15, wherein the psychiatric disorder comprises depression.

17. A method for treating dopamine related dysfunction in a mammal comprising administering to the mammal a dopamine reuptake inhibiting amount of a boat tropane selected from the following compounds:
2β-(1-Propanoyl)-3α-(4-fluorophenyl)-tropane;
2β-(1-Propanoyl)-3α-(3,4-dichlorophenyl)tropane;
1-{3α-(3,4-Dichlorophenyl)-7β-hydroxy-8-methyl-8-azabicyclo{3.2.1}oct-2-yl}propan-1-one;
2β-Carbomethoxy-3α-(3,4-dichlorophenyl)-6β-hydroxy-8-methyl-8-azabicyclo{3.2.1}octane;
2β-Carbomethoxy-3α-(2-naphthyl)-6β-hydroxy-8-methyl-8-azabicyclo{3.2.1}octane;
2β-Carbomethoxy-3α-(4-fluorophenyl)-6β-hydroxy-8-methyl-8-azabicyclo{3.2.1}octane;
2β-Carbomethoxy-3α-phenyl-6β-hydroxy-8-methyl-8-azabicyclo{3.2.1}octane;
2β-Carbomethoxy-3α-(3,4-dichlorophenyl)-7β-hydroxy-8-methyl-8-azabicyclo{3.2.1}octane;
(1S)-2β-Carbomethoxy-3α-(3,4-dichlorophenyl)-7β-hydroxy-8-methyl-8-azabicyclo{3.2.1}octane;
(1R)-2β-Carbomethoxy-3α-(3,4-dichlorophenyl)-7β-hydroxy-8-methyl-8-azabicyclo{3.2.1}octane;
2β-Carbomethoxy-3α-(2-naphthyl)-7β-hydroxy-8-methyl-8-azabicyclo{3.2.1}octane;
2β-Carbomethoxy-3α-(4-fluorophenyl)-7β-hydroxy-8-methyl-8-azabicyclo{3.2.1}octane;
2β-Carbomethoxy-3α-phenyl-7β-hydroxy-8-methyl-8-azabicyclo{3.2.1}octane;
2β-Carbomethoxy-3α-(3,4-dichlorophenyl)-7α-benzoyloxy-8-methyl-8-azabicyclo{3.2.1}octane;
2β-Carbomethoxy-3α-(3,4-dichlorophenyl)-6α-benzoyloxy-8-methyl-8-azabicyclo{3.2.1}octane;
2β-Carbomethoxy-3α-(3,4-dichlorophenyl)-7α-hydroxy-8-methyl-8-azabicyclo{3.2.1}octane;
2β-Carbomethoxy-3α-(3,4-dichlorophenyl)- 6α-hydroxy-8-methyl-8-azabicyclo{3.2.1}octane;
2β-Carbomethoxy-3α-(3,4-dichlorophenyl)-8-methyl-8-azabicyclo{3.2.1}oct-7-one;
2β-Carbomethoxy-3β-(3,4-dichlorophenyl)-8-methyl-8-azabicyclo{3.2.1}oct-7-one;
2β-Carbomethoxy-3α-bis(fluorophenyl)methoxy-7β-hydroxy-8-methyl-8-azabicyclo{3.2.1}octane; and
2β-Carbomethoxy-3α-bis(4-fluorophenyl)methoxy-6β-hydroxy-8-methyl-8-azabicyclo{3.2.1}octane.

18. A method for treating dopamine related dysfunction in a mammal comprising administering to the mammal a dopamine reuptake inhibiting amount of a compound selected from the following compounds:
2β-(1-Propanoyl)-3α-(4-fluorophenyl)-tropane;
2β-(1-Propanoyl)-3α-(3,4-dichlorophenyl)tropane;
1-{3α-(3,4-Dichlorophenyl)-7β-hydroxy-8-methyl-8-azabicyclo{3.2.1}oct-2-yl}propan-1-one;
2β-Carbomethoxy-3α-(3,4-dichlorophenyl)-6β-hydroxy-8-methyl-8-azabicyclo{3.2.1}octane;
2β-Carbomethoxy-3α-(2-naphthyl)-6β-hydroxy-8-methyl-8-azabicyclo{3.2.1}octane;
2β-Carbomethoxy-3α-(4-fluorophenyl)-6β-hydroxy-8-methyl-8-azabicyclo{3.2.1}octane;
2β-Carbomethoxy-3α-phenyl-6β-hydroxy-8-methyl-8-azabicyclo{3.2.1}octane;
2β-Carbomethoxy-3α-(3,4-dichlorophenyl)-7β-hydroxy-8-methyl-8-azabicyclo{3.2.1}octane;
(1S)-2β-Carbomethoxy-3α-(3,4-dichlorophenyl)-7β-hydroxy-8-methyl-8-azabicyclo{3.2.1}octane;
(1R)-2β-Carbomethoxy-3α-(3,4-dichlorophenyl)-7β-hydroxy-8-methyl-8-azabicyclo{3.2.1}octane;
2β-Carbomethoxy-3α-(2-naphthyl)-7β-hydroxy-8-methyl-8-azabicyclo{3.2.1}octane;
2β-Carbomethoxy-3α-(4-fluorophenyl)-762 -hydroxy-8-methyl-8-azabicyclo{3.2.1}octane;
2β-Carbomethoxy-3α-phenyl-7β-hydroxy-8-methyl-8-azabicyclo{3.2.1}octane;
2β-Carbomethoxy-3α-(3,4-dichlorophenyl)-7α-benzoyloxy-8-methyl-8-azabicyclo{3.2.1}octane;
2β-Carbomethoxy-3α-(3,4-dichlorophenyl)-6α-benzoyloxy-8-methyl-8-azabicyclo{3.2.1}octane;
2β-Carbomethoxy-3α-(3,4-dichlorophenyl)-7α-hydroxy-8-methyl-8-azabicyclo{3.2.1}octane;
2β-Carbomethoxy-3α-(3,4-dichlorophenyl)-6α-hydroxy-8-methyl-8-azabicyclo{3.2.1}octane;
2β-Carbomethoxy-3α-(3,4-dichlorophenyl)-8-methyl-8-azabicyclo{3.2.1}oct-7-one;
2β-Carbomethoxy-3β-(3,4-dichlorophenyl)-8-methyl-8-azabicyclo{3.2.1}oct-7-one;
2β-Carbomethoxy-3α-bis(fluorophenyl)methoxy-7β-hydroxy-8-methyl-8-azabicyclo{3.2.1}octane; and
2β-Carbomethoxy-3α-bis(4-fluorophenyl)methoxy-6β-hydroxy-8-methyl-8-azabicyclo{3.2.1}octane.

19. The method according to claim 18, wherein the dopamine related dysfunction comprises Attention deficit disorder.

20. A method for treating cocaine abuse in a mammal comprising administering to the mammal an effective amount of a compound selected from the following compounds:
2β-(1-Propanoyl)-3α-(4-fluorophenyl)-tropane;
2β-(1-Propanoyl)-3α-(3,4-dichlorophenyl)tropane;
1-{3α-(3,4-Dichlorophenyl)-7β-hydroxy-8-methyl-8-azabicyclo{3.2.1}oct-2-yl}propan-1-one;
2β-Carbomethoxy-3α-(3,4-dichlorophenyl)-6β-hydroxy-8-methyl-8-azabicyclo{3.2.1}octane;
2β-Carbomethoxy-3α-(2-naphthyl)-6β-hydroxy-8-methyl-8-azabicyclo{3.2.1}octane;
2β-Carbomethoxy-3α-(4-fluorophenyl)-6β-hydroxy-8-methyl-8-azabicyclo{3.2.1}octane;
2β-Carbomethoxy-3α-phenyl-6β-hydroxy-8-methyl-8-azabicyclo{3.2.1}octane;
2β-Carbomethoxy-3α-(3,4-dichlorophenyl)-7β-hydroxy-8-methyl-8-azabicyclo{3.2.1}octane;
(1S)-2β-Carbomethoxy-3α-(3,4-dichlorophenyl)-7β-hydroxy-8-methyl-8-azabicyclo{3.2.1}octane;
(1R)-2β-Carbomethoxy-3α-(3,4-dichlorophenyl)-7β-hydroxy-8-methyl-8-azabicyclo{3.2.1}octane;
2β-Carbomethoxy-3α-(2-naphthyl)-7β-hydroxy-8-methyl-8-azabicyclo{3.2.1}octane;
2β-Carbomethoxy-3α-(4-fluorophenyl)-7β-hydroxy-8-methyl-8-azabicyclo{3.2.1}octane;
2β-Carbomethoxy-3α-phenyl-7β-hydroxy-8-methyl-8-azabicyclo{3.2.1}octane;
2β-Carbomethoxy-3α-(3,4-dichlorophenyl)-7α-benzoyloxy-8-methyl-8-azabicyclo{3.2.1}octane;
2β-Carbomethoxy-3α-(3,4-dichlorophenyl)-6α-benzoyloxy-8-methyl-8-azabicyclo{3.2.1}octane;
2β-Carbomethoxy-3α-(3,4-dichlorophenyl)-7α-hydroxy-8-methyl-8-azabicyclo{3.2.1}octane;
2β-Carbomethoxy-3α-(3,4-dichlorophenyl)-6α-hydroxy-8-methyl-8-azabicyclo{3.2.1}octane;
2β-Carbomethoxy-3α-(3,4-dichlorophenyl)-8-methyl-8-azabicyclo{3.2.1}oct-7-one;
2β-Carbomethoxy-3β-(3,4-dichlorophenyl)-8-methyl-8-azabicyclo{3.2.1}oct-7-one;
2β-Carbomethoxy-3α-bis(fluorophenyl)methoxy-7β-hydroxy-8-methyl-8-azabicyclo{3.2.1}octane; and
2β-Carbomethoxy-3α-bis(4-fluorophenyl)methoxy-6β-hydroxy-8-methyl-8-azabicyclo{3.2.1}octane.

21. A method for treating clinical dysfunction in a mammal comprising administering to the mammal an effective amount of a compound selected from the following compounds:
2β-(1-Propanoyl)-3α-(4-fluorophenyl)-tropane;
2β-(1-Propanoyl)-3α-(3,4-dichlorophenyl)tropane;
1-{3α-(3,4-Dichlorophenyl)-7β-hydroxy-8-methyl-8-azabicyclo{3.2.1}oct-2-yl}propan-1-one;
2β-Carbomethoxy-3α-(3,4-dichlorophenyl)-6β-hydroxy-8-methyl-8-azabicyclo{3.2.1}octane;
2β-Carbomethoxy-3α-(2-naphthyl)-6β-hydroxy-8-methyl-8-azabicyclo{3.2.1}octane;
2β-Carbomethoxy-3α-(4-fluorophenyl)-6 β-hydroxy-8-methyl-8-azabicyclo{3.2.1}octane;
2β-Carbomethoxy-3α-phenyl-6β-hydroxy-8-methyl-8-azabicyclo{3.2.1}octane;
2β-Carbomethoxy-3α-(3,4-dichlorophenyl)-7β-hydroxy-8-methyl-8-azabicyclo{3.2.1}octane;
(1S)-2β-Carbomethoxy-3α-(3,4-dichlorophenyl)-7β-hydroxy-8-methyl-8-azabicyclo{3.2.1}octane;
(1R)-2β-Carbomethoxy-3α-(3,4-dichlorophenyl)-7β-hydroxy-8-methyl-8-azabicyclo{3.2.1}octane;
2β-Carbomethoxy-3α-(2-naphthyl)-7β-hydroxy-8-methyl-8-azabicyclo{3.2.1}octane;
2β-Carbomethoxy-3α-(4-fluorophenyl)-7β-hydroxy-8-methyl-8-azabicyclo{3.2.1}octane;
2β-Carbomethoxy-3α-phenyl-7β-hydroxy-8-methyl-8-azabicyclo{3.2.1}octane;
2β-Carbomethoxy-3α-(3,4-dichlorophenyl)-7α-benzoyloxy-8-methyl-8-azabicyclo{3.2.1}octane;
2β-Carbomethoxy-3α-(3,4-dichlorophenyl)-6α-benzoyloxy-8-methyl-8-azabicyclo{3.2.1}octane;
2β-Carbomethoxy-3α-(3,4-dichlorophenyl)-7α-hydroxy-8-methyl-8-azabicyclo{3.2.1}octane;
2β-Carbomethoxy-3α-(3,4-dichlorophenyl)-6α-hydroxy-8-methyl-8-azabicyclo{3.2.1}octane;
2β-Carbomethoxy-3α-(3,4-dichlorophenyl)-8-methyl-8-azabicyclo{3.2.1}oct-7-one;
2β-Carbomethoxy-3β-(3,4-dichlorophenyl)-8-methyl-8-azabicyclo{3.2.1}oct-7-one;
2β-Carbomethoxy-3α-bis(fluorophenyl)methoxy-7β-hydroxy-8-methyl-8-azabicyclo{3.2.1}octane; and
2β-Carbomethoxy-3α-bis(4-fluorophenyl)methoxy-6β-hydroxy-8-methyl-8-azabicyclo{3.2.1}octane.

22. The method of claim 21, wherein the clinical dysfunction comprises migraine.
